(19) United States
(12) Patent Application Publication
(10) Pub. No.: US 2014/0005145 A1
(43) Pub. Date: Jan. 2, 2014

(54) COMBINATION BREAST CANCER THERAPY WITH HSP90 INHIBITORY COMPOUNDS

(71) Applicant: David Proia, Newton, MA (US)

(72) Inventor: David Proia, Newton, MA (US)

(73) Assignee: SYNTA PHARMACEUTICALS CORP., Lexington, MA (US)

(21) Appl. No.: 13/992,591

(22) PCT Filed: Dec. 7, 2011
(86) PCT No.: PCT/US11/63742

§ 371 Date: Jul. 23, 2013

Related U.S. Application Data
(60) Provisional application No. 61/420,920, filed on Dec. 8, 2010.

(51) Int. Cl. A61K 31/675; A61K 31/4196; A61K 31/138; A61K 31/565

(52) U.S. Cl. CPC A61K 31/675; A61K 31/565; A61K 31/4196; A61K 31/138
USPC 514/80; 514/171; 514/384

(57) ABSTRACT

Methods for treating breast cancer, comprising administering to the subject an effective amount of a selective estrogen receptor modulator and an effective amount of a compound represented by the following structural formula: (I) (Ia) a tautomer, or a pharmaceutically acceptable salt thereof. The variables depicted in the structural formula are defined herein.

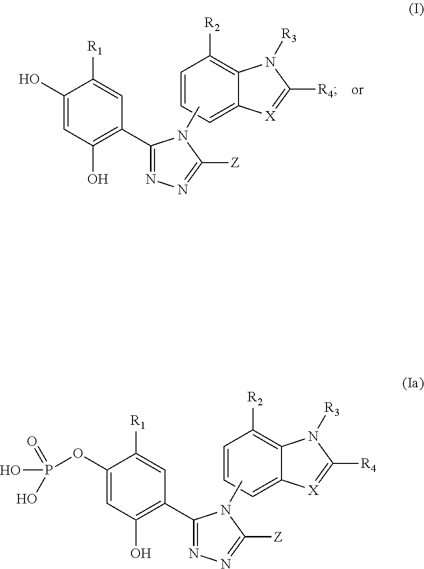

### BACKGROUND OF THE INVENTION

Breast cancer is the most common type of non-skin cancer in women and the fifth most common cause of cancer death. Breast cancer is usually treated with surgery and then possibly with chemotherapy or radiation, or both. Hormone positive breast cancers are treated with long term hormone blocking therapy. However, a major limitation to the efficacy of hormonal therapies in the management of advanced breast cancers is the frequent development of acquired resistance. As such, there is a continuing need for therapies that can be used to treat hormonal therapy resistant breast cancers.

### SUMMARY OF THE INVENTION

It is now found that certain triazolone HSP90 inhibitor and selective estrogen receptor modulator combinations are surprisingly effective at treating subjects with hormonal therapy resistant breast cancer, particularly tamoxifen resistant breast cancer without further increasing side effects. The particular therapies disclosed herein demonstrate surprising biological activity by demonstrating significant anticancer effects while showing minimal side effects.

The present invention utilizes triazolone compounds which inhibit the activity of HSP90 and are useful in the treatment of breast cancer, particularly metastatic, or hormonal therapy resistant breast cancer in combination with selective estrogen receptor modulators such as tamoxifen, raloxifene, toremifene, fulvestrant, megestrol acetate, fluoxymesterone, and ethinyl estradiol. A method of treating a subject with breast cancer includes the step of administering to the subject an HSP90 inhibitor described herein and a selective estrogen receptor modulator. In one embodiment, the method is to treat a subject who is being or has been treated with a selective estrogen receptor modulator with an HSP90 inhibitor and a selective estrogen receptor modulator. In one embodiment, the administration of the HSP90 inhibitor and the selective estrogen receptor modulator are done concurrently. In another embodiment, the administration of the HSP90 inhibitor and the selective estrogen receptor modulator are done separately. In another embodiment, the administration of the HSP90 inhibitor and the selective estrogen receptor modulator are done sequentially. In another embodiment, the HSP90 inhibitor and the selective estrogen receptor modulator are administered in a single formulation. In another embodiment, the HSP90 inhibitor and the selective estrogen receptor modulator are administered intravenously, orally, transdermally, subcutaneously, mucosally, intramuscularly, intranasally, intrapulmonarily, parenterally, intrarectally or topically. In any one of these embodiments, the selective estrogen receptor modulator is tamoxifen, raloxifene, toremifene, fulvestrant, megestrol acetate, fluoxymesterone, or ethinyl estradiol. In any one of these embodiments, the HSP90 inhibitor is a compound represented by a compound of formulae (I) or (Ia), or a compound in Table 1 or 2.

In one embodiment, the invention includes the use of an HSP90 inhibitor described herein for the manufacture of a medicament for treating breast cancer in combination with a selective estrogen receptor modulator.

In certain embodiments, the combination treatment utilizing an HSP90 compound described herein with other chemotherapeutic agents may help to prevent or reduce the development of drug resistant breast cancer, particularly metastatic or hormonal therapy resistant breast cancer. In this embodiment, the compounds of the invention may allow a reduced efficacious amount of a second chemotherapeutic agent given to a mammal, because the HSP90 inhibitor should inhibit the development of drug resistant breast cancer such as hormonal therapy resistant, particularly tamoxifen resistant breast cancer. In one embodiment, the second chemotherapeutic agent is a selective estrogen receptor modulator. In another embodiment, the selective estrogen receptor modulator is tamoxifen, raloxifene, toremifene, fulvestrant, megestrol acetate, fluoxymesterone, or ethinyl estradiol.

Selective estrogen receptor modulators (SERMs) are a class of compounds that act on estrogen receptor. Typical members of SERMs include, but not limited to, tamoxifen, raloxifene, toremifene, fulvestrant, megestrol acetate, fluoxymesterone, and ethinyl estradiol. SERMs find various therapeutic uses depending on their pattern of action in different tissues. For example, tamoxifen and toremifene are used in the treatment of breast cancer, and raloxifene is used in the treatment of breast cancer and osteoporosis. To examine the anticancer effect of HSP90 inhibitors of the invention, such as Compound 1, in combination with selective estrogen receptor modulators, in vitro studies and in vivo studies were conducted. These study results demonstrated that Compound 1 showed surprising synergistic effect in combination with tamoxifen in the treatment of breast cancer in the preclinical model.

### BRIEF DESCRIPTION OF THE FIGURE

FIG. 1 shows a nude mouse xenograft study conducted to determine the effect of Compound 1 on the in vivo growth rate of MCF-7 human breast cancer tumor cells when dosed in combination with tamoxifen. Tumor-bearing animals (5 mice/group) were i.v. injected 1 time per week for a total of 3 doses with 10 mL/kg of 10/18 DRD vehicle with or without 180 mg/kg Compound 1. Animals were also implanted with slow release tamoxifen pellets (Tam). In the combination treatment group, animals were dosed with Compound 1 four days after tamoxifen pellets were implanted. Each group that was not dosed with Compound 1 was instead dosed with 10/18 DRD vehicle as a mock treatment. Average tumor volumes for each group were determined every 3-5 days. Treatment with a combination of slow release tamoxifen, in combination with 180 mg/kg Compound 1, dosed one time per week, induced significant tumor regression relative to that achieved by either single therapy alone.

### DETAILED DESCRIPTION OF THE INVENTION

Unless otherwise specified, the below terms used herein are defined as follows: As used herein, the term “alkyl” means a saturated, straight chain or branched, non-cyclic hydrocarbon having from 1 to 10 carbon atoms. Representative straight chain alkyls include methyl, ethyl, n-propyl, n-butyl, n-pentyl, n-hexyl, n-heptyl, n-octyl, n-nonyl and n-decyl; while representative branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl, and the like. The term “(C₁-C₆)alkyl” means a saturated, straight chain or branched, non-cyclic hydrocarbon having from 1 to 6 carbon atoms. Alkyl groups included in compounds of this invention may be optionally substituted with one or more substituents.

As used herein, the term “alkenyl” means a straight chain or branched, non-cyclic hydrocarbon having from 2 to 10 carbon atoms and having at least one carbon-carbon double bond. Representative straight chain and branched (C₂-C₁₀)alkenyls include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, 3-decenyl, and the like. Alkenyl groups included in compounds of the invention may be optionally substituted with one or more substituents.

As used herein, the term “alkynyl” means a straight chain or branched, non-cyclic hydrocarbon having from 2 to 10 carbon atoms and having at least one carbon-carbon triple bond. Representative straight chain and branched alkynyls include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like. Alkynyl groups included in compounds of the invention may be optionally substituted with one or more substituents.

As used herein, the term “cycloalkyl” means a saturated, mono- or polycyclic, non-aromatic hydrocarbon having from 3 to 20 carbon atoms. Representative cycloalkyls include cyclopropyl, 1-methylcyclopropyl, cyclobutyl, cyclopentyl, cyclohexyl, cycloheptyl, cyclooctyl, cyclononyl, cyclodecyl, octahydropentalenyl, and the like. Cycloalkyl groups included in compounds of the invention may be optionally substituted with one or more substituents.

As used herein, the term “cycloalkenyl” means a mono- or polycyclic, non-aromatic hydrocarbon having at least one carbon-carbon double bond in the cyclic system and having from 3 to 20 carbon atoms. Representative cycloalkenyls include cyclopentenyl, cyclopentadienyl, cyclohexenyl, cyclohexadienyl, cycloheptenyl, cycloheptadienyl, cycloheptatrienyl, cyclooctenyl, cyclooctadienyl, cyclooctatrienyl, cyclooctatetraenyl, cyclononenyl, cyclononadienyl, cyclodecenyl, cyclodecadienyl, 1,2,3,4,5,8-hexahydronaphthalenyl, and the like. Cycloalkenyl groups included in compounds of the invention may be optionally substituted with one or more substituents.

As used herein, the term “alkylene” refers to an alkyl group that has two points of attachment. The term “(C₁-C₆)alkylene” refers to an alkylene group that has from one to six carbon atoms. Straight chain (C₁-C₆)alkylene groups are preferred. Non-limiting examples of alkylene groups include methylene (—CH₂—), ethylene (—CH₂CH₂—), n-propylene (—CH₂CH₂CH₂—), isopropylene (—CH₂CH(CH₃)—), and the like. Alkylene groups included in compounds of this invention may be optionally substituted with one or more substituents.

As used herein, the term “lower” refers to a group having up to four atoms. For example, a “lower alkyl” refers to an alkyl radical having from 1 to 4 carbon atoms, “lower alkoxy” refers to “—O—(C₁-C₄)alkyl and a “lower alkenyl” or “lower alkynyl” refers to an alkenyl or alkynyl radical having from 2 to 4 carbon atoms.

As used herein, the term “haloalkyl” means an alkyl group, in which one or more, including all, the hydrogen radicals are replaced by a halo group(s), wherein each halo group is independently selected from —F, —Cl, —Br, and —I. For example, the term “halomethyl” means a methyl in which one to three hydrogen radical(s) have been replaced by a halo group. Representative haloalkyl groups include trifluoromethyl, bromomethyl, 1,2-dichloroethyl, 4-iodobutyl, 2-fluoropentyl, and the like.

As used herein, an “alkoxy” is an alkyl group which is attached to another moiety via an oxygen linker. Alkoxy groups included in compounds of this invention may be optionally substituted with one or more substituents.

As used herein, a “haloalkoxy” is a haloalkyl group which is attached to another moiety via an oxygen linker.

As used herein, the term an “aromatic ring” or “aryl” means a mono- or polycyclic hydrocarbon, containing from 6 to 15 carbon atoms, in which at least one ring is aromatic. Examples of suitable aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Aryl groups included in compounds of this invention may be optionally substituted with one or more substituents. In one embodiment, the aryl group is a monocyclic ring, wherein the ring comprises 6 carbon atoms, referred to herein as “(C₆)aryl.”

As used herein, the term “aralkyl” means an aryl group that is attached to another group by a (C₁-C₆)alkylene group. Representative aralkyl groups include benzyl, 2-phenyl-ethyl, naphth-3-yl-methyl and the like. Aralkyl groups included in compounds of this invention may be optionally substituted with one or more substituents.

As used herein, the term “heterocyclyl” means a monocyclic or a polycyclic, saturated or unsaturated, non-aromatic ring or ring system which typically contains 5- to 20-members and at least one heteroatom. A heterocyclic ring system can contain saturated ring(s) or unsaturated non-aromatic ring(s), or a mixture thereof. A 3- to 10-membered heterocycle can contain up to 5 heteroatoms, and a 7- to 20-membered heterocycle can contain up to 7 heteroatoms. Typically, a heterocycle has at least one carbon atom ring member. Each heteroatom is independently selected from nitrogen, which can be oxidized (e.g., N(O)) or quaternized, oxygen and sulfur, including sulfoxide and sulfone. The heterocycle may be attached via any heteroatom or carbon atom. Representative heterocycles include morpholinyl, thiomorpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl, and the like. A heteroatom may be substituted with a protecting group known to those of ordinary skill in the art, for example, a nitrogen atom may be substituted with a tert-butoxycarbonyl group. Furthermore, the heterocyclyl included in compounds of this invention may be optionally substituted with one or more substituents. Only stable isomers of such substituted heterocyclic groups are contemplated in this definition.

As used herein, the term “heteroaromatic”, “heteroaryl”, or like terms, means a monocyclic or a polycyclic, unsaturated radical containing at least one heteroatom, in which at least one ring is aromatic. Polycyclic heteroaryl rings must contain at least one heteroatom, but not all rings of a polycyclic heteroaryl moiety must contain heteroatoms. Each heteroatom is independently selected from nitrogen, which can be oxidized (e.g., N(O)) or quaternized, oxygen and sulfur, including sulfoxide and sulfone. Representative heteroaryl groups include pyridyl, 1-oxo-pyridyl, furanyl, benzo[1,3]dioxolyl, benzo[1,4]dioxinyl, thienyl, pyrrolyl, oxazolyl, imidazolyl, thiazolyl, a isoxazolyl, quinolinyl, pyrazolyl, isothiazolyl, pyridazinyl, pyrimidinyl, pyrazinyl, a triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzothienyl. In one embodiment, the heteroaromatic ring is selected from 5-8 membered monocyclic heteroaryl rings. The point of attachment of a heteroaromatic or heteroaryl ring may be at either a carbon atom or a heteroatom. Heteroaryl groups included in compounds of this invention may be optionally substituted with one or more substituents. As used herein, the term “(C₅)heteroaryl” means an heteroaromatic ring of 5 members, wherein at least one carbon atom of the ring is replaced with a heteroatom, such as, for example, oxygen, sulfur or nitrogen. Representative (C₅)heteroaryls include furanyl, thienyl, pyrrolyl, oxazolyl, imidazolyl, thiazolyl, isoxazolyl, pyrazolyl, isothiazolyl, pyrazinyl, triazolyl, thiadiazolyl, and the like. As used herein, the term “(C₆)heteroaryl” means an aromatic heterocyclic ring of 6 members, wherein at least one carbon atom of the ring is replaced with a heteroatom such as, for example, oxygen, nitrogen or sulfur. Representative (C6)heteroaryls include pyridyl, pyridazinyl, pyrazinyl, triazinyl, tetrazinyl, and the like.

As used herein, the term “heteroaralkyl” means a heteroaryl group that is attached to another group by a (C₁-C₆)alkylene. Representative heteroaralkyls include 2-(pyridin-4-yl)-propyl, 2-(thien-3-yl)-ethyl, imidazol-4-yl-methyl, and the like. Heteroaralkyl groups included in compounds of this invention may be optionally substituted with one or more substituents.

As used herein, the term “halogen” or “halo” means —F, —Cl, —Br or —I.

As used herein the term “heteroalkyl” means a straight or branched alkyl group wherein one or more of the internal carbon atoms in the chain is replaced by a heteroatom. For example, a heteroalkyl is represented by the formula —[CH₂]ₓ—Z-[CH₂]_{y}[CH₃], wherein x is a positive integer and y is zero or a positive integer, Z is O, NR, S, S(O), or S(O)₂, and wherein replacement of the carbon atom does not result in a unstable compound. Heteroalkyl groups included in compounds of this invention may be optionally substituted with one or more substituents.

Suitable substituents for an alkyl, alkylene, alkenyl, alkynyl, cycloalkyl, cycloalkenyl, heterocyclyl, aryl, aralkyl, heteroaryl, and heteroaralkyl groups include are those substituents which form a stable compound of the invention without significantly adversely affecting the reactivity or biological activity of the compound of the invention. Examples of substituents for an alkyl, alkylene, alkenyl, alkynyl, cycloalkyl, cycloalkenyl, heterocyclyl, aryl, aralkyl, heteroaryl, and heteroaralkyl include an alkyl, alkenyl, alkynyl, cycloalkyl, cycloalkenyl, heterocyclyl, aryl, heteroaryl, aralkyl, heteraralkyl, heteroalkyl, alkoxy, (each of which can be optionally and independently substituted), —C(O)NR²⁸R²⁹, —C(S)NR²⁸R²⁹, —C(NR³²)NR²⁸R²⁹, —NR³³C(O)R³¹, —NR³³C(S)R³¹, —NR³³C(NR³²)R³¹, halo, —OR³³₅ cyano, nitro, —C(O)R³³, —C(S)R³³, —C(NR³²)R³³, —NR²⁸R²⁹, —C(O)OR³³, —C(S)OR³³, —C(NR³²)OR³³, —OC(O)R³³, —OC(S)R³³, —OC(NR³²)R³³, —NR³⁰C(O)NR²⁸R²⁹, —NR³³C(S)NR²⁸R²⁹, —NR³³C(NR³²)NR²⁸R²⁹, —OC(O)NR²⁸R²⁹, —OC(S)NR²⁸R²⁹, —OC(NR³²)NR²⁸R²⁹, —NR³³C(O)OR³¹, —NR³³C(S)OR³¹, —NR³³C(NR³²)OR³¹, —S(O)ₚR³³, —OS(O)ₚR³³, —NR³³S(O)ₚR³³, —S(O)ₚNR²⁸R²⁹, —OS(O)ₚNR²⁸R²⁹, —NR³³S(O)ₚNR²⁸R²⁹, guanidino, —C(O)SR³¹, —C(S)SR³¹, —C(NR³²)SR³¹, —OC(O)OR³¹, —OC(S)OR³¹, —OC(NR³²)OR³¹, —SC(O)R³³, —SC(O)R³¹, —SC(NR³²)OR³¹, —SC(S)R³³, —SC(S)OR³¹, —SC(O)NR²⁸R²⁹, —SC(NR³²)NR²⁸R²⁹, —SC(S)NR²⁸R²⁹, —SC(NR³²)R³³, —OS(O)ₚOR³¹, —S(O)ₚOR³¹, —NR³⁰S(O)ₚOR³¹, —SS(O)ₚR³³, —SS(O)ₚOR³¹, —SS(O)ₚNR²⁸R²⁹, —OP(O)(OR³¹)₂, or —SP(O)(OR³¹)₂. In addition, any saturated portion of an alkyl, cycloalkyl, alkylene, heterocyclyl, alkenyl, cycloalkenyl, alkynyl, aralkyl and heteroaralkyl groups, may also be substituted with ═O, ═S, or ═N—R³².

Each R²⁸ and R²⁹ is independently H, alkyl, alkenyl, alkynyl, cycloalkyl, cycloalkenyl, heterocyclyl, aryl, heteroaryl, aralkyl, or heteraralkyl, wherein each alkyl, alkenyl, alkynyl, cycloalkyl, cycloalkenyl, heterocyclyl, aryl, heteroaryl, aralkyl, or heteroalkyl represented by R²⁸ or R²⁹ is optionally and independently substituted.

Each R³¹ and R³³ is independently H, alkyl, alkenyl, alkynyl, cycloalkyl, cycloalkenyl, heterocyclyl, aryl, heteroaryl, aralkyl, or heteraralkyl, wherein each alkyl, alkenyl, alkynyl, cycloalkyl, cycloalkenyl, heterocyclyl, aryl, heteroaryl, aralkyl, and heteraralkyl represented by R³¹ or R³³ is optionally and independently unsubstituted.

Each R³² is independently H, alkyl, alkenyl, alkynyl, cycloalkyl, cycloalkenyl, heterocyclyl, aryl, heteroaryl, aralkyl, heteraralkyl, —C(O)R³³, —C(O)NR²⁸R²⁹, —S(O)ₚR³³, or —S(O)NR²⁸R²⁹, wherein each alkyl, alkenyl, alkynyl, cycloalkyl, cycloalkenyl, heterocyclyl, aryl, heteroaryl, aralkyl and heteraralkyl represented by R³² is optionally and independently substituted.

The variable p is 0, 1 or 2.

When a heterocyclyl, heteroaryl or heteroaralkyl group contains a nitrogen atom, it may be substituted or unsubstituted. When a nitrogen atom in the aromatic ring of a heteroaryl group has a substituent, the nitrogen may be oxidized or a quaternary nitrogen.

As used herein, the terms “subject”, “patient” and “mammal” are used interchangeably. The terms “subject” and “patient” refer to an animal (e.g., a bird such as a chicken, quail or turkey, or a mammal), preferably a mammal including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

As used herein, the term “compound(s) of this invention” “triazolone compound”, or similar terms refers to a compound of any one of formulae (I) or (Ia) or a compound in Table 1 or 2, or a pharmaceutically acceptable salt thereof.

As used herein, the term “selective estrogen receptor modulator” or “SERM” refers to a class of compounds that act on the estrogen receptor. SERMs typically include, but not limited to, such compounds as tamoxifen, raloxifene, toremifene, fulvestrant, megestrol acetate, fluoxymesterone, and ethinyl estradiol.

As used herein, the term “metastatic breast cancer” refers to breast cancer when it spreads to the other parts of the body of a subject, while the term “hormonal therapy resistant breast cancer” or “hormone resistant breast cancer” refers to breast cancer that is not responsive to hormonal therapy or has developed resistance after treatment of hormonal therapy in a subject.

Some of the disclosed methods can be particularly effective at treating subjects whose cancer has become “drug resistant” or “multi-drug resistant”. A cancer which initially responded to an anti-cancer drug becomes resistant to the anti-cancer drug when the anti-cancer drug is no longer effective in treating the subject with the cancer. For example, many tumors will initially respond to treatment with an anti-cancer drug by decreasing in size or even going into remission, only to develop resistance to the drug. “Drug resistant” tumors are characterized by a resumption of their growth and/or reappearance after having seemingly gone into remission, despite the administration of increased dosages of the anti-cancer drug. Cancers that have developed resistance to two or more anti-cancer drugs are said to be “multi-drug resistant”. For example, it is common for cancers to become resistant to three or more anti-cancer agents, often five or more anti-cancer agents and at times ten or more anti-cancer agents.

As used herein, the term “pharmaceutically acceptable salt” refers to a salt prepared from a compound of any one of formulae (I) or (Ia) or a compound in Table 1 or 2 having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl, N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. The term “pharmaceutically acceptable salt” also refers to a salt prepared from a compound of any one of formulae (I) or (Ia) or a compound in Table 1 or 2 having a basic functional group, such as an amine functional group, and a pharmaceutically acceptable inorganic or organic acid. Suitable acids include, but are not limited to, hydrogen sulfate, citric acid, acetic acid, oxalic acid, hydrochloric acid (HCl), hydrogen bromide (HBr), hydrogen iodide (HI), nitric acid, hydrogen bisulfide, phosphoric acid, isonicotinic acid, oleic acid, tannic acid, pantothenic acid, saccharic acid, lactic acid, salicylic acid, tartaric acid, bitartratic acid, ascorbic acid, succinic acid, maleic acid, besylic acid, fumaric acid, gluconic acid, glucaronic acid, formic acid, benzoic acid, glutamic acid, methanesulfonic acid, ethanesulfonic acid, benzenesulfonic acid, pamoic acid and p-toluenesulfonic acid.

A pharmaceutically acceptable carrier may contain inert ingredients which do not unduly inhibit the biological activity of the compound(s). The pharmaceutically acceptable carriers should be biocompatible, i.e., non-toxic, non-inflammatory, non-immunogenic and devoid of other undesired reactions upon the administration to a subject. Standard pharmaceutical formulation techniques can be employed, such as those described in REMINGTON, J. P., REMINGTON'S PHARMACEUTICAL SCIENCES (Mack Pub. Co., 17^{th} ed., 1985). Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate, and the like. Methods for encapsulating compositions, such as in a coating of hard gelatin or cyclodextran, are known in the art. See BAKER, ET AL., CONTROLLED RELEASE OF BIOLOGICAL ACTIVE AGENTS, (John Wiley and Sons, 1986).

As used herein, the term “effective amount” refers to an amount of a compound of this invention which is sufficient to reduce or ameliorate the severity, duration, progression, or onset of a disease or disorder, delay onset of a disease or disorder, retard or halt the advancement of a disease or disorder, cause the regression of a disease or disorder, prevent or delay the recurrence, development, onset or progression of a symptom associated with a disease or disorder, or enhance or improve the therapeutic effect(s) of another therapy. The precise amount of compound administered to a subject will depend on the mode of administration, the type and severity of the disease or condition and on the characteristics of the subject, such as general health, age, sex, body weight and tolerance to drugs. For example, for a proliferative disease or disorder, determination of an effective amount will also depend on the degree, severity and type of cell proliferation. The skilled artisan will be able to determine appropriate dosages depending on these and other factors. When co-administered with other therapeutic agents, e.g., when co-administered with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of the invention being used. In cases where no amount is expressly noted, an effective amount should be assumed. Non-limiting examples of an effective amount of a compound of the invention are provided herein below. In a specific embodiment, the invention provides a method of treating, managing, or ameliorating breast cancer, particularly metastatic, or hormonal therapy resistant breast cancer, or one or more symptoms thereof, said method comprising administering to a subject in need thereof a dose of at least 150 μg/kg, at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds of the invention once every day, once every 2 days, once every 3 days, once every 4 days, once every 5 days, once every 6 days, once every 7 days, once every 8 days, once every 10 days, once every two weeks, once every three weeks, or once a month. The daily dose can be administered in a single portion. Alternatively, the daily dose can be divided into portions (typically equal portions) administered two times, three times, four times or more per day.

The dosage of a therapeutic agent other than a compound of the invention, which has been or is currently being used to treat, manage, or ameliorate breast cancer, particularly metastatic, or hormonal therapy resistant breast cancer, or one or more symptoms thereof, can be used in the combination therapies of the invention. Preferably, the dosage of each individual therapeutic agent used in said combination therapy is lower than the dose of an individual therapeutic agent when given independently to treat, manage, or ameliorate a disease or disorder, or one or more symptoms thereof. The recommended dosages of therapeutic agents currently used for the treatment, management, or amelioration of a disease or disorder, or one or more symptoms thereof, can obtained from any reference in the art. See, e.g., GOODMAN & GILMAN'S THE PHARMACOLOGICAL BASIS OF BASIS OF THERAPEUTICS 9^{TH} ED, (Hardman, et al., Eds., NY: Mc-Graw-Hill (1996)); PHYSICIAN'S DESK REFERENCE 57^{TH} ED. (Medical Economics Co., Inc., Montvale, N.J. (2003)).

As used herein, the terms “treat”, “treatment” and “treating” refer to the reduction or amelioration of the progression, severity and/or duration of a disease or disorder, delay of the onset of a disease or disorder, or the amelioration of one or more symptoms (preferably, one or more discernible symptoms) of a disease or disorder, resulting from the administration of one or more therapies (e.g., one or more therapeutic agents such as a compound of the invention). The terms “treat”, “treatment” and “treating” also encompass the reduction of the risk of developing a disease or disorder, and the delay or inhibition of the recurrence of a disease or disorder. In specific embodiments, the terms “treat”, “treatment” and “treating” refer to the amelioration of at least one measurable physical parameter of a disease or disorder, such as growth of a tumor, not necessarily discernible by the patient. In other embodiments the terms “treat”, “treatment” and “treating” refer to the inhibition of the progression of a disease or disorder, e.g., breast cancer, metastatic breast cancer, or hormonal therapy resistant cancer, either physically by the stabilization of a discernible symptom, physiologically by the stabilization of a physical parameter, or both. In another embodiment, the terms “treat”, “treatment” and “treating” of a proliferative disease or disorder refers to the reduction or stabilization of tumor size or cancerous cell count, and/or delay of tumor formation.

As used herein, the terms “therapeutic agent” and “therapeutic agents” refer to any agent(s) that can be used in the treatment of a disease or disorder, e.g. breast cancer, metastatic breast cancer, or hormonal therapy resistant breast cancer, or one or more symptoms thereof. In certain embodiments, the term “therapeutic agent” refers to a compound of the invention. In certain other embodiments, the term “therapeutic agent” does not refer to a compound of the invention. Preferably, a therapeutic agent is an agent that is known to be useful for, or has been or is currently being used for the treatment of a disease or disorder, e.g., breast cancer, metastatic breast cancer, or hormonal therapy resistant breast cancer, or one or more symptoms thereof.

As used herein, the term “synergistic” refers to a combination of a compound of the invention and another therapeutic agent, which, when taken together, is more effective than the additive effects of the individual therapies. A synergistic effect of a combination of therapies (e.g., a combination of therapeutic agents) permits the use of lower dosages of one or more of the therapeutic agent(s) and/or less frequent administration of said agent(s) to a subject with a disease or disorder, e.g., breast cancer, metastatic breast cancer, or hormonal therapy resistant breast cancer. The ability to utilize lower dosage of one or more therapeutic agent and/or to administer said therapeutic agent less frequently reduces the toxicity associated with the administration of said agent to a subject without reducing the efficacy of said therapy in the treatment of a disease or disorder. In addition, a synergistic effect can result in improved efficacy of agents in the prevention, management or treatment of a disease or disorder, e.g. breast cancer, metastatic breast cancer, or hormonal therapy resistant breast cancer. Finally, a synergistic effect of a combination of therapies may avoid or reduce adverse or unwanted side effects associated with the use of either therapeutic agent alone.

As used herein, the term “in combination” refers to the use of more than one therapeutic agent. The use of the term “in combination” does not restrict the order in which said therapeutic agents are administered to a subject with breast cancer, metastatic breast cancer, or hormonal therapy resistant breast cancer. A first therapeutic agent, such as a compound of the invention, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent, such as an anti-cancer agent, to a subject with breast cancer, metastatic breast cancer, or hormonal therapy resistant breast cancer.

As used herein, the terms “therapies” and “therapy” can refer to any protocol(s), method(s), and/or agent(s) that can be used in the prevention, treatment, management, or amelioration of breast cancer, metastatic breast cancer, or hormonal therapy resistant breast cancer.

A used herein, a “protocol” includes dosing schedules and dosing regimens. The protocols herein are methods of use and include therapeutic protocols.

As used herein, a composition that “substantially” comprises a compound means that the composition contains more than about 80% by weight, more preferably more than about 90% by weight, even more preferably more than about 95% by weight, and most preferably more than about 97% by weight of the compound.

The compounds of the invention are defined herein by their chemical structures and/or chemical names. Where a compound is referred to by both a chemical structure and a chemical name, and the chemical structure and chemical name conflict, the chemical structure is determinative of the compound's identity.

Only those choices and combinations of substituents that result in a stable structure are contemplated. Such choices and combinations will be apparent to those of ordinary skill in the art and may be determined without undue experimentation.

The invention can be understood more fully by reference to the following detailed description and illustrative examples, which are intended to exemplify non-limiting embodiments of the invention.

The selective estrogen receptor modulators employed in the disclosed invention include, but not limited to, tamoxifen, raloxifene, toremifene, fulvestrant, megestrol acetate, fluoxymesterone, and ethinyl estradiol. Tamoxifen is currently used for the treatment of both early and advanced estrogen receptor positive breast cancer in pre- and post-menopause women. Additionally, it is the most common hormone treatment for male breast cancer. The chemical structures of some selective estrogen receptor modulators are shown below:

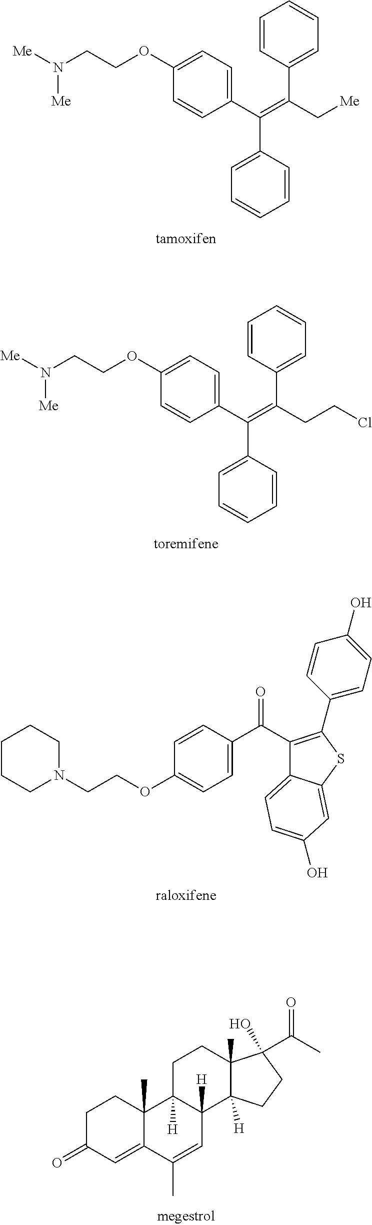

The present invention utilizes selective estrogen receptor modulators and compounds represented by Formulae (I) or (Ia):

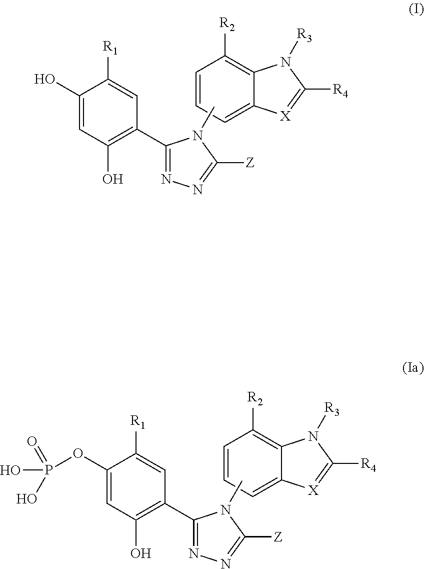

or a tautomer, or a pharmaceutically acceptable salt thereof, wherein:
Z is OH, SH, or NHR₇;
X is CR₄ or N;
R₁ is —H, —OH, —SH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanadino, a haloalkyl, a heteroalkyl, an alkoxy or cycloalkoxy, a haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂;
R₂ is —H, —OH, —SH, —NR₇H, —OR₁₅, —SR₁₅, —NHR₁₅, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, or —NR₇C(NR₈)NR₁₀R₁₁;
R₃ is —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, a haloalkyl, a heteroalkyl, —C(O)R₇, —(CH₂)ₘC(O)OR₇, —C(O)OR₇, —OC(O)R₇, —C(O)NR₁₀R₁₁, —S(O)ₚR₇, —S(O)ₚOR₇, or —S(O)ₚNR₁₀R₁₁;
R₄ is —H, —OH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, halo, cyano, nitro, guanadino, a haloalkyl, a heteroalkyl, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, —S(O)ₚNR₁₀R₁₁, or R₄₃ and R₄₄ taken together with the carbon atoms to which they are attached form an optionally substituted cycloalkenyl, an optionally substituted aryl, an optionally substituted heterocyclyl, or an optionally substituted heteroaryl;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;
R₁₅, for each occurrence, is independently, a lower alkyl;
p, for each occurrence, is, independently, 1 or 2; and
m, for each occurrence, is independently, 1, 2, 3, or 4.

In one embodiment, in formula (I) or (Ia), X is CR₄.

In another embodiment, in formula (I) or (Ia), X is N.

In another embodiment, in formula (I) or (Ia), R₁ is selected from the group consisting of —H, lower alkyl, lower alkoxy, lower cycloalkyl, and lower cycloalkoxy.

In another embodiment, in formula (I) or (Ia), R₁ is selected from the group consisting of —H, methyl, ethyl, propyl, isopropyl, cyclopropyl, methoxy, ethoxy, propoxy, and cyclopropoxy.

In another embodiment, in formula (I) or (Ia), R₃ is selected from the group consisting of —H, a lower alkyl, a lower cycloalkyl, —C(O)N(R₂₇)₂, and —C(O)OH, wherein R₂₇ is —H or a lower alkyl.

In another embodiment, in formula (I) or (Ia), R₃ is selected from the group consisting of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, and —C(O)N(CH₃)₂.

In one embodiment, R₄ is H or a lower alkyl.

In another embodiment, in formula (I) or (Ia), R₄ is selected from the group consisting of —H, methyl, ethyl, propyl, isopropyl or cyclopropyl.

In another embodiment, in formula (I) or (Ia), R₁ is selected from the group consisting of —H, —OH, —SH, —NH₂, a lower alkoxy and a lower alkyl amino.

In another embodiment, in formula (I) or (Ia), R₁ is selected from the group consisting of —H, —OH, methoxy and ethoxy.

In another embodiment, in formula (I) or (Ia), Z is —OH.

In another embodiment, in formula (I) or (Ia), Z is —SH.

In another embodiment, in formula (I) or (Ia), R₂ is selected from the group consisting of —H, —OH, —SH, —NH₂, a lower alkoxy and a lower alkyl amino.

In another embodiment, in formula (I) or (Ia), R₂ is selected from the group consisting of —H, —OH, methoxy, and ethoxy.

In another embodiment, the compound is selected from the group consisting of:
3-(2,4-dihydroxy-5-ethyl-phenyl)-4-(1,3-dimethyl-indol-5-yl)-5-hydroxy-[1,2,4]triazole,
3-(2,4-dihydroxy-5-isopropyl-phenyl)-4-(1,3-dimethyl-indol-5-yl)-5-hydroxy-[1,2,4]triazole,
3-(2,4-dihydroxy-5-isopropyl-phenyl)-4-(1-methyl-indol-5-yl)-5-hydroxy-[1,2,4]triazole,
3-(2,4-dihydroxy-5-isopropyl-phenyl)-4-(1-isopropyl-indol-4-yl)-5-hydroxy-[1,2,4]triazole,
3-(2,4-dihydroxy-5-isopropyl-phenyl)-4-(1-methyl-indazol-5-yl)-5-mercapto-[1,2,4]triazole,
3-(2,4-dihydroxy-5-isopropyl-phenyl)-4-(1-methyl-indazol-6-yl)-5-mercapto-[1,2,4]triazole,
3-(2,4-dihydroxyphenyl)-4-(1-ethyl-indol-4-yl)-5-mercapto-[1,2,4]triazole,
3-(2,4-dihydroxyphenyl)-4-(1-isopropyl-indol-4-yl)-5-mercapto-[1,2,4]triazole,
3-(2,4-dihydroxyphenyl)-4-(indol-4-yl)-5-mercapto-[1,2,4]triazole,
3-(2,4-dihydroxyphenyl)-4-(1-methoxyethyl-indol-4-yl)-5-mercapto-[1,2,4]triazole,
3-(2,4-dihydroxy-5-ethyl-phenyl)-4-(1-isopropyl-indol-4-yl)-5-mercapto-[1,2,4]triazole,
3-(2,4-dihydroxyphenyl)-4-(1-dimethylcarbamoyl-indol-4-yl)-5-mercapto-[1,2,4]triazole,
3-(2,4-dihydroxy-5-ethyl-phenyl)-4-(1-propyl-indol-4-yl)-5-mercapto-[1,2,4]triazole,
3-(2,4-dihydroxy-5-ethyl-phenyl)-4-(1,2,3-trimethyl-indol-5-yl)-5-mercapto-[1,2,4]triazole,
3-(2,4-dihydroxy-5-ethyl-phenyl)-4-(2,3-dimethyl-indol-5-yl)-5-mercapto-[1,2,4]triazole,
3-(2,4-dihydroxy-5-ethyl-phenyl)-4-(1-acetyl-2,3-dimethyl-indol-5-yl)-5-mercapto-[1,2,4]triazole,
3-(2,4-dihydroxy-5-ethyl-phenyl)-4-(1-propyl-2,3-dimethyl-indol-5-yl)-5-mercapto-[1,2,4]triazole,
3-(2,4-dihydroxy-5-ethyl-phenyl)-4-(1-n-butyl-indol-4-yl)-5-mercapto-[1,2,4]triazole,
3-(2,4-dihydroxy-5-ethyl-phenyl)-4-(1-n-pentyl-indol-4-yl)-5-mercapto-[1,2,4]triazole,
3-(2,4-dihydroxy-5-ethyl-phenyl)-4-(1-n-hexyl-indol-4-yl)-5-mercapto-[1,2,4]triazole,
3-(2,4-dihydroxy-5-cyclopropyl-phenyl)-4-(1-(1-methylcyclopropyl)-indol-4-yl)-5-mercapto-[1,2,4]triazole,
3-(2,4-dihydroxy-5-cyclopropyl-phenyl)-4-(1,2,3-trimethyl-indol-5-yl)-5-mercapto-[1,2,4]triazole,
3-(2,4-dihydroxy-5-ethyl-phenyl)-4-(1-methyl-3-ethyl-indol-5-yl)-5-mercapto-[1,2,4]triazole,
3-(2,4-dihydroxy-5-ethyl-phenyl)-4-(1,3-dimethyl-indol-5-yl)-5-mercapto-[1,2,4]triazole,
3-(2,4-dihydroxy-5-ethyl-phenyl)-4-(1-methyl-3-isopropyl-indol-5-yl)-5-mercapto-[1,2,4]triazole,
3-(2,4-dihydroxy-5-ethyl-phenyl)-4-(1,2-dimethyl-indol-5-yl)-5-mercapto-[1,2,4]triazole,
3-(2,4-dihydroxy-5-ethyl-phenyl)-4-(N-methyl-indol-5-yl)-5-mercapto-[1,2,4]triazole,
3-(2,4-dihydroxy-5-isopropyl-phenyl)-4-(1,3-dimethyl-indol-5-yl)-5-mercapto-[1,2,4]triazole,
3-(2,4-dihydroxy-5-cyclopropyl-phenyl)-4-(1,3-dimethyl-indol-5-yl)-5-mercapto-[1,2,4]triazole,
3-(2,4-dihydroxy-5-cyclopropyl-phenyl)-4-(1-methyl-indol-5-yl)-5-mercapto-[1,2,4]triazole,
3-(2,4-dihydroxy-5-isopropyl-phenyl)-4-(1H-indol-5-yl)-5-mercapto-[1,2,4]triazole,
3-(2,4-dihydroxy-5-ethyl-phenyl)-4-(1,2-dimethyl-indol-5-yl)-5-mercapto-[1,2,4]triazole,
3-(2,4-dihydroxy-5-isopropyl-phenyl)-4-(1-ethyl-indol-5-yl)-5-mercapto-[1,2,4]triazole,
3-(2,4-dihydroxy-5-isopropyl-phenyl)-4-(1-propyl-indol-5-yl)-5-mercapto-[1,2,4]triazole, or a tautomer, or a pharmaceutically acceptable salt thereof.

In another embodiment, in formula (I) or (Ia), X is N.

In another embodiment, the compound is selected from the group consisting of
3-(2,4-dihydroxy-5-ethyl-phenyl)-4-(1-ethyl-benzimidazol-4-yl)-5-mercapto-[1,2,4]triazole,
3-(2,4-dihydroxy-5-ethyl-phenyl)-4-(1-ethyl-benzimidazol-4-yl)-5-mercapto-[1,2,4]triazole HCL salt,
3-(2,4-dihydroxy-5-ethyl-phenyl)-4-(2-methyl-3-ethyl-benzimidazol-5-yl)-5-mercapto-[1,2,4]triazole,
3-(2,4-dihydroxy-5-ethyl-phenyl)-4-(1-ethyl-2-methyl-benzimidazol-5-yl)-5-mercapto-[1,2,4]triazole,
3-(2,4-dihydroxy-5-isopropyl-phenyl)-4-(1-methyl-2-trifluoromethyl-benzimidazol-5-yl)-5-mercapto-[1,2,4]triazole, or a tautomer, or a pharmaceutically acceptable salt thereof.

### i) Exemplary Compounds of the Invention

Exemplary compounds of the invention are depicted in Table 1 below, including tautomers or pharmaceutically acceptable salts.

TABLE 1

| | Structure | Tautomeric Structure | Name |
|---|---|---|---|
| 1 | 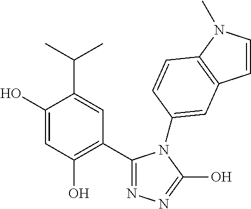 [SMILES: CC(C)c1cc(-c2nnc(O)n2-c2ccc3c(ccn3C)c2)c(O)cc1O] | 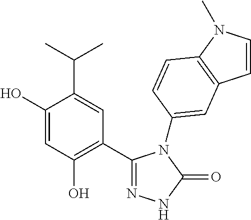 [SMILES: CC(C)c1cc(-c2n[nH]c(=O)n2-c2ccc3c(ccn3C)c2)c(O)cc1O] | 3-(2,4-dihydroxy-5- isopropyl-phenyl)-4- (1-methyl-indol-5-yl)- 5-hydroxy-[1,2,4] triazole |
| 2 | 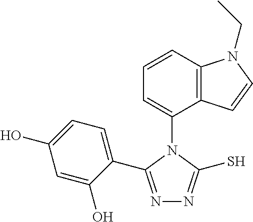 [SMILES: CCn1ccc2c(-n3c(S)nnc3-c3ccc(O)cc3O)cccc21] | 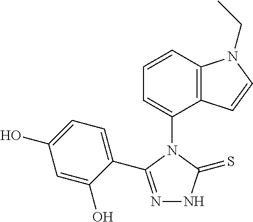 [SMILES: CCn1ccc2c(-n3c(-c4ccc(O)cc4O)n[nH]c3=S)cccc21] | 3-(2,4- Dihydroxyphenyl)-4- (1-ethyl-indol-4-yl)-5- mercapto-[1,2,4] triazole |
| 3 | 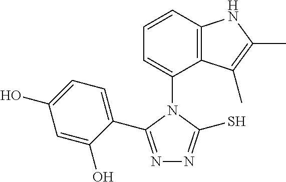 [SMILES: Cc1[nH]c2cccc(-n3c(S)nnc3-c3ccc(O)cc3O)c2c1C] | 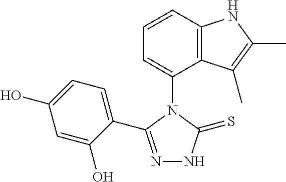 [SMILES: Cc1[nH]c2cccc(-n3c(-c4ccc(O)cc4O)n[nH]c3=S)c2c1C] | 3-(2,4-Dihydroxy- phenyl)-4-(2,3- dimethyl-1H-indol-4- yl)-5-mercapto-[1,2,4] triazole |
| 4 | 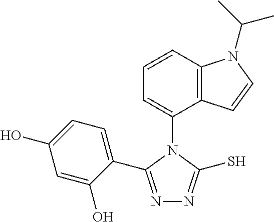 [SMILES: CC(C)n1ccc2c(-n3c(S)nnc3-c3ccc(O)cc3O)cccc21] | 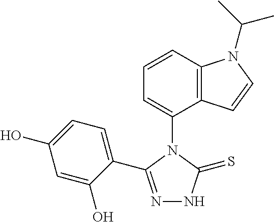 [SMILES: CC(C)n1ccc2c(-n3c(-c4ccc(O)cc4O)n[nH]c3=S)cccc21] | 3-(2,4- Dihydroxyphenyl)-4- (1-isopropyl-indol-4- yl)-5-mercapto-[1,2,4] triazole |
| 5 | 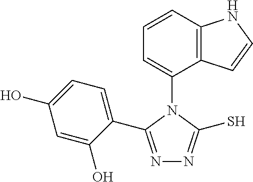 [SMILES: Oc1ccc(-c2nnc(S)n2-c2cccc3[nH]ccc23)c(O)c1] | 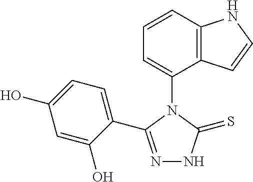 [SMILES: Oc1ccc(-c2n[nH]c(=S)n2-c2cccc3[nH]ccc23)c(O)c1] | 3-(2,4-Dihydroxy- phenyl)-4-(indol-4- yl)-5-mercapto-[1,2,4] triazole |
| 6 | 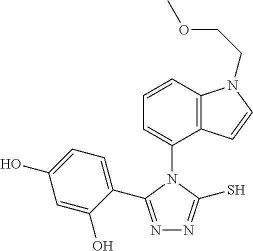 [SMILES: COCCn1ccc2c(-n3c(S)nnc3-c3ccc(O)cc3O)cccc21] | 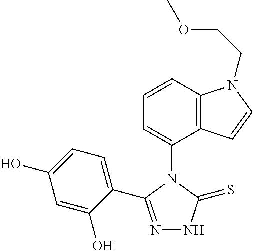 [SMILES: COCCn1ccc2c(-n3c(-c4ccc(O)cc4O)n[nH]c3=S)cccc21] | 3-(2,4-Dihydroxy- phenyl)-4-[1-(2- methoxyethoxy)- indol-4-yl]-5- mercapto-[1,2,4] triazole |
| 7 | 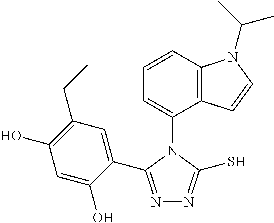 [SMILES: CCc1cc(-c2nnc(S)n2-c2cccc3c2ccn3C(C)C)c(O)cc1O] | 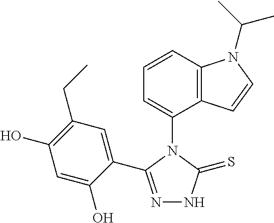 [SMILES: CCc1cc(-c2n[nH]c(=S)n2-c2cccc3c2ccn3C(C)C)c(O)cc1O] | 3-(2,4-Dihydroxy-5- ethyl-phenyl)-4-(1- isopropyl-indol-4-yl)- 5-mercapto-[1,2,4] triazole |
| 8 | 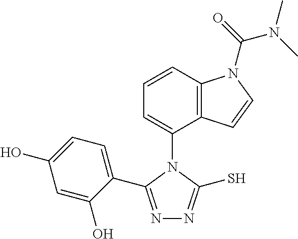 [SMILES: CN(C)C(=O)n1ccc2c(-n3c(S)nnc3-c3ccc(O)cc3O)cccc21] | 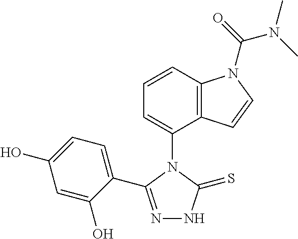 [SMILES: CN(C)C(=O)n1ccc2c(-n3c(-c4ccc(O)cc4O)n[nH]c3=S)cccc21] | 3-(2,4-Dihydroxy-5- ethyl-phenyl)-4-[1- (dimethyl- carbamoyl)-indol-4- yl]-5-mercapto-[1,2,4] triazole |
| 9 | 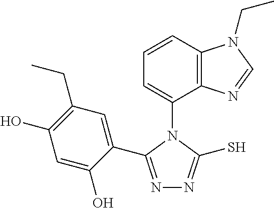 [SMILES: CCc1cc(-c2nnc(S)n2-c2cccc3c2ncn3CC)c(O)cc1O] | 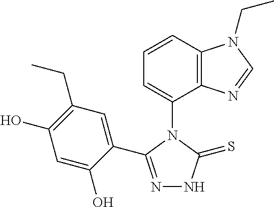 [SMILES: CCc1cc(-c2n[nH]c(=S)n2-c2cccc3c2ncn3CC)c(O)cc1O] | 3-(2,4-Dihydroxy-5- ethyl-phenyl)-4-(1- ethyl-benzoimidazol- 4-yl)-5-mercapto- [1,2,4]triazole |
| 10 | 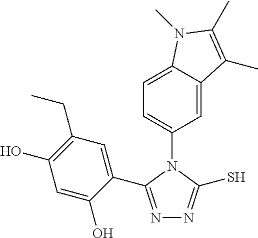 [SMILES: CCc1cc(-c2nnc(S)n2-c2ccc3c(c2)c(C)c(C)n3C)c(O)cc1O] | 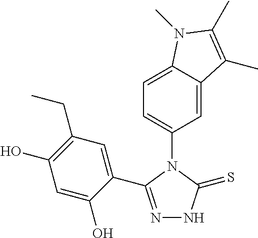 [SMILES: CCc1cc(-c2n[nH]c(=S)n2-c2ccc3c(c2)c(C)c(C)n3C)c(O)cc1O] | 3-(2,4-Dihydroxy-5- ethyl-phenyl)-4- (1,2,3-trimethyl-indol- 5-yl)-5-mercapto- [1,2,4]triazole |
| 11 | 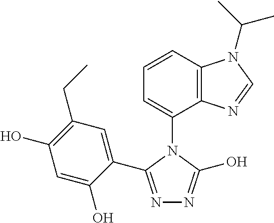 [SMILES: CCc1cc(-c2nnc(O)n2-c2cccc3c2ncn3C(C)C)c(O)cc1O] | 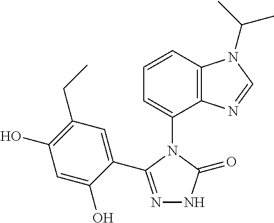 [SMILES: CCc1cc(-c2n[nH]c(=O)n2-c2cccc3c2ncn3C(C)C)c(O)cc1O] | 3-(2,4-Dihydroxy-5- ethyl-phenyl)-4-(1- isopropyl-indol-3-yl)- 5-hydroxy-[1,2,4] triazole |
| 12 | 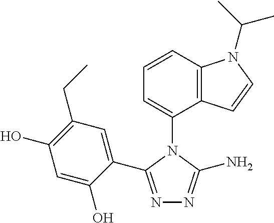 [SMILES: CCc1cc(-c2nnc(N)n2-c2cccc3c2ccn3C(C)C)c(O)cc1O] | 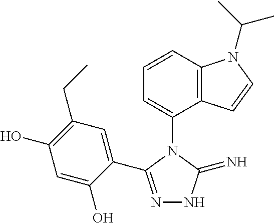 [SMILES: CCc1cc(-c2n[nH]c(=N)n2-c2cccc3c2ccn3C(C)C)c(O)cc1O] | 3-(2,4-Dihydroxy-5- ethyl-phenyl)-4-(1- isopropyl-indol-4-yl)- 5-amino-[1,2,4] triazole |
| 15 | 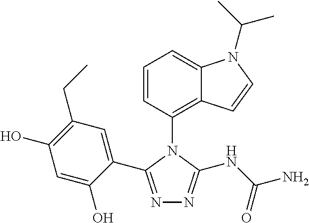 [SMILES: CCc1cc(-c2nnc(NC(N)=O)n2-c2cccc3c2ccn3C(C)C)c(O)cc1O] | | 3-(2,4-Dihydroxy-5- ethyl-phenyl)-4-(1- isopropyl-indol-4-yl)- 5-ureido-[1,2,4] triazole |
| 16 | 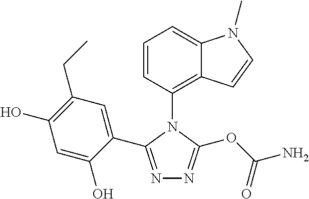 [SMILES: CCc1cc(-c2nnc(OC(N)=O)n2-c2cccc3c2ccn3C)c(O)cc1O] | | 3-(2,4-Dihydroxy-5- ethyl-phenyl)-4-(1- methyl-indol-4-yl)-5- carbamoyloxy-[1,2,4] triazole |
| 17 | 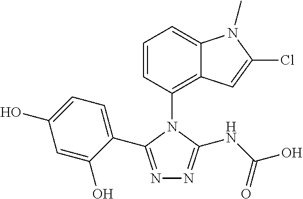 [SMILES: Cn1c(Cl)cc2c(-n3c(NC(=O)O)nnc3-c3ccc(O)cc3O)cccc21] | | 3-(2,4-Dihydroxy- phenyl)-4-(1-methyl- 2-chloro-indol-4-yl)- 5-carbamoyloxy- [1,2,4]triazole |
| 18 | 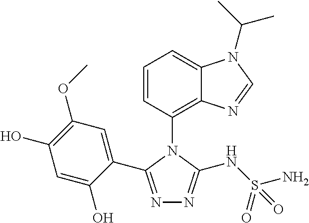 [SMILES: COc1cc(-c2nnc(NS(N)(=O)=O)n2-c2cccc3c2ncn3C(C)C)c(O)cc1O] | | 3-(2,4-Dihydroxy-5- methoxy-phenyl)-4- (1-isopropyl- benzoimidazol-4-yl)- 5-(sulfamoylamino)- [1,2,4]triazole |
| 20 | 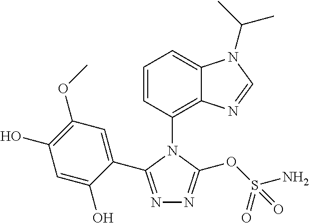 [SMILES: COc1cc(-c2nnc(OS(N)(=O)=O)n2-c2cccc3c2ncn3C(C)C)c(O)cc1O] | | 3-(2,4-Dihydroxy-5- methoxy-phenyl)-4- (1-isopropyl- benzoimidazol-4-yl)- 5-(sulfamoyloxy)- [1,2,4]triazole |
| 21 | 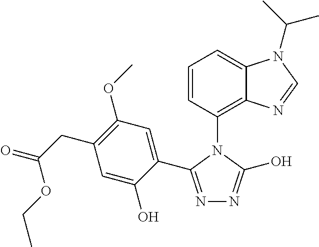 [SMILES: CCOC(=O)Cc1cc(O)c(-c2nnc(O)n2-c2cccc3c2ncn3C(C)C)cc1OC] | 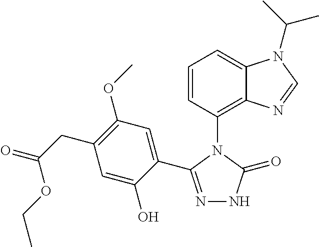 [SMILES: CCOC(=O)Cc1cc(O)c(-c2n[nH]c(=O)n2-c2cccc3c2ncn3C(C)C)cc1OC] | 3-(2-Hydroxy-4- ethoxycarbonyoxy-5- methoxy-phenyl)-4- (1-isopropyl- benzoimidazol-4-yl)- 5-hydroxy-[1,2,4] triazole |
| 22 | 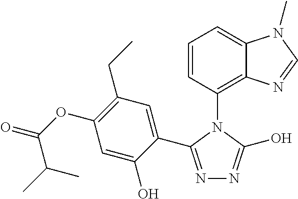 [SMILES: CCc1cc(-c2nnc(O)n2-c2cccc3c2ncn3C)c(O)cc1OC(=O)C(C)C] | 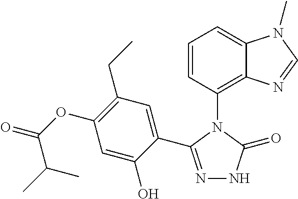 [SMILES: CCc1cc(-c2n[nH]c(=O)n2-c2cccc3c2ncn3C)c(O)cc1OC(=O)C(C)C] | 3-[2-Hydroxy-4- isobutyryloxy-5-ethyl- phenyl]-4-(1-methyl- benzo-imidazol-4-yl)- 5-hydroxy-[1,2,4] triazole |
| 23 | 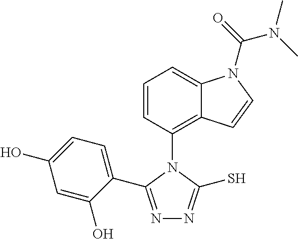 [SMILES: CN(C)C(=O)n1ccc2c(-n3c(S)nnc3-c3ccc(O)cc3O)cccc21] | 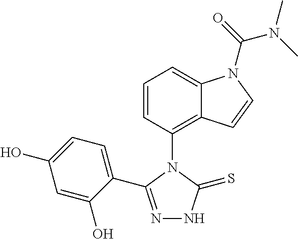 [SMILES: CN(C)C(=O)n1ccc2c(-n3c(-c4ccc(O)cc4O)n[nH]c3=S)cccc21] | 3-(2,4-Dihydroxy- phenyl)-4-(1- dimethylcarbamoyl- indol-4-yl)-5- mercapto-[1,2,4] triazole |
| 24 | 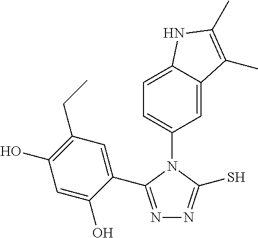 [SMILES: CCc1cc(-c2nnc(S)n2-c2ccc3[nH]c(C)c(C)c3c2)c(O)cc1O] | 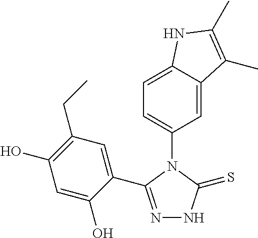 [SMILES: CCc1cc(-c2n[nH]c(=S)n2-c2ccc3[nH]c(C)c(C)c3c2)c(O)cc1O] | 3-(2,4-Dihydroxy-5- ethyl-phenyl)-4-(2,3- dimethyl-indol-5-yl)- 5-mercapto-[1,2,4] triazole |
| 25 | 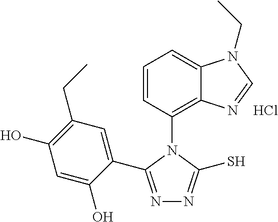 [SMILES: CCc1cc(-c2nnc(S)n2-c2cccc3c2ncn3CC)c(O)cc1O ; Cl] | 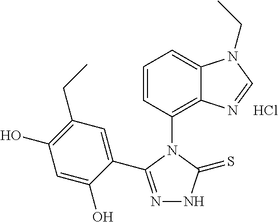 [SMILES: CCc1cc(-c2n[nH]c(=S)n2-c2cccc3c2ncn3CC)c(O)cc1O ; Cl] | 3-(2,4-Dihydroxy-5- ethyl-phenyl)-4-(1- ethyl-1H- benzoimidazol-4-yl)- 5-mercapto-[1,2,4] triazole, HCl salt |
| 26 | 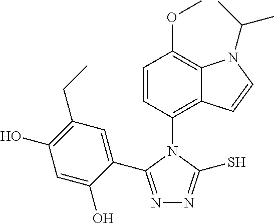 [SMILES: CCc1cc(-c2nnc(S)n2-c2ccc(OC)c3c2ccn3C(C)C)c(O)cc1O] | 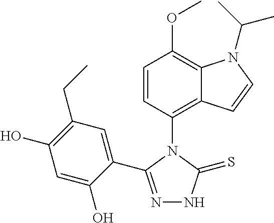 [SMILES: CCc1cc(-c2n[nH]c(=S)n2-c2ccc(OC)c3c2ccn3C(C)C)c(O)cc1O] | 3-(2,4-Dihydroxy-5- ethyl-phenyl)-4-(1- isopropyl-7-methoxy- indol-4-yl)-5- mercapto-[1,2,4] triazole |
| 27 | 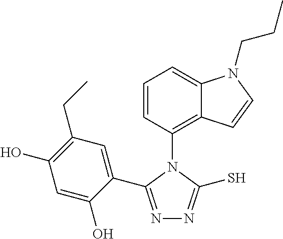 [SMILES: CCCn1ccc2c(-n3c(S)nnc3-c3cc(CC)c(O)cc3O)cccc21] | 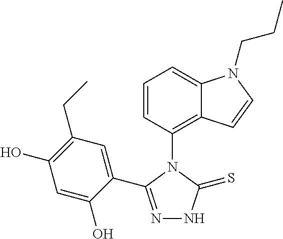 [SMILES: CCCn1ccc2c(-n3c(-c4cc(CC)c(O)cc4O)n[nH]c3=S)cccc21] | 3-(2,4-dihydroxy-5- ethyl-phenyl)-4-(1- propyl-indol-4-yl)-5- mercapto-[1,2,4] triazole |
| 28 | 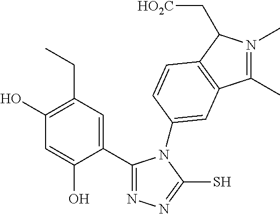 | 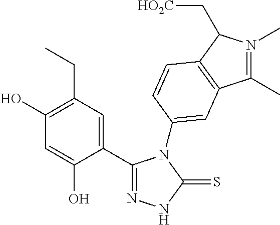 | 3-(2,4-dihydroxy-5- ethyl-phenyl)-4-(1- acetyl-2,3-dimehyl- indol-5-yl)-5- mercapto-[1,2,4] triazole |
| 29 | 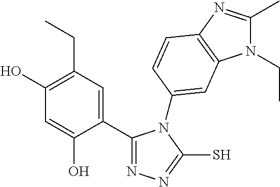 [SMILES: CCc1cc(-c2nnc(S)n2-c2ccc3nc(C)n(CC)c3c2)c(O)cc1O] | 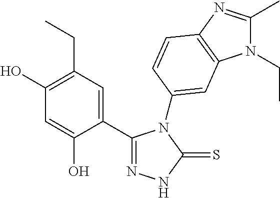 [SMILES: CCc1cc(-c2n[nH]c(=S)n2-c2ccc3nc(C)n(CC)c3c2)c(O)cc1O] | 3-(2,4-dihydroxy-5- ethyl-phenyl)-4-(2- methyl-3-ethyl- benzimidazol-5-yl)-5- mercapto-[1,2,4] triazole |
| 30 | 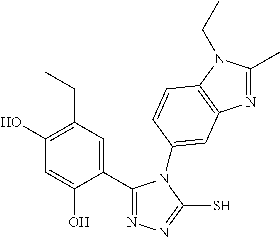 [SMILES: CCc1cc(-c2nnc(S)n2-c2ccc3c(c2)nc(C)n3CC)c(O)cc1O] | 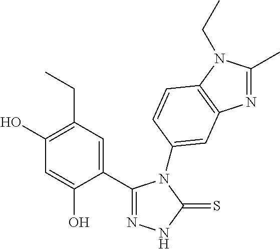 [SMILES: CCc1cc(-c2n[nH]c(=S)n2-c2ccc3c(c2)nc(C)n3CC)c(O)cc1O] | 3-(2,4-dihydroxy-5- ethyl-phenyl)-4-(1- ethyl-2-methyl- benzimidazol-5-yl)-5- mercapto-[1,2,4] triazole |
| 31 | 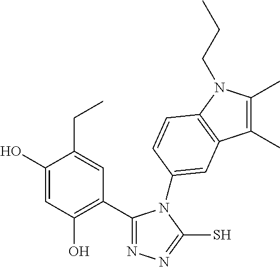 [SMILES: CCCn1c(C)c(C)c2cc(-n3c(S)nnc3-c3cc(CC)c(O)cc3O)ccc21] | 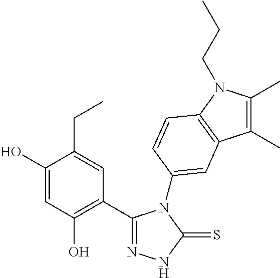 [SMILES: CCCn1c(C)c(C)c2cc(-n3c(-c4cc(CC)c(O)cc4O)n[nH]c3=S)ccc21] | 3-(2,4-dihydroxy-5- ethyl-phenyl)-4-(1- propyl-2,3-dimethyl- indol-5-yl)-5- mercapto-[1,2,4] triazole |
| 34 | 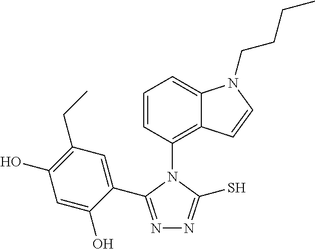 [SMILES: CCCCn1ccc2c(-n3c(S)nnc3-c3cc(CC)c(O)cc3O)cccc21] | 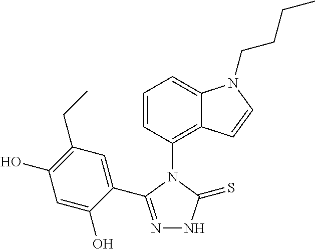 [SMILES: CCCCn1ccc2c(-n3c(-c4cc(CC)c(O)cc4O)n[nH]c3=S)cccc21] | 3-(2,4-dihydroxy-5- ethyl-phenyl)-4-(1-n- butyl-indol-4-yl)-5- mercapto-[1,2,4] triazole |
| 35 | 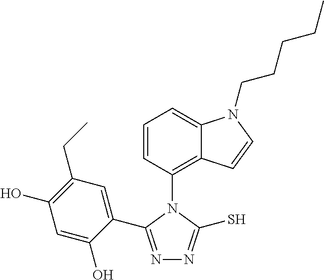 [SMILES: CCCCCn1ccc2c(-n3c(S)nnc3-c3cc(CC)c(O)cc3O)cccc21] | 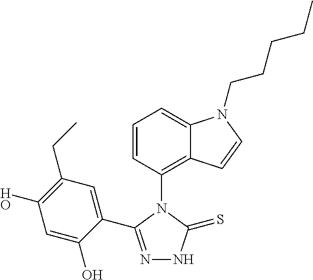 [SMILES: CCCCCn1ccc2c(-n3c(-c4cc(CC)c(O)cc4O)n[nH]c3=S)cccc21] | 3-(2,4-dihydroxy-5- ethyl-phenyl)-4-(1-n- pentyl-indol-4-yl)-5- mercapto-[1,2,4] triazole |
| 36 | 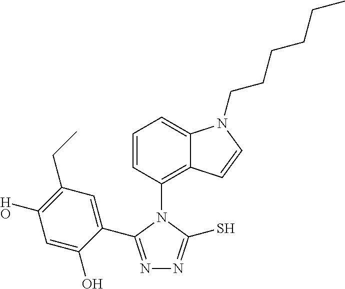 [SMILES: CCCCCCn1ccc2c(-n3c(S)nnc3-c3cc(CC)c(O)cc3O)cccc21] | 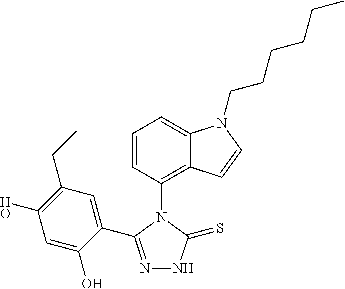 [SMILES: CCCCCCn1ccc2c(-n3c(-c4cc(CC)c(O)cc4O)n[nH]c3=S)cccc21] | 3-(2,4-dihydroxy-5- ethyl-phenyl)-4-(1-n- hexyl-indol-4-yl)-5- mercapto-[1,2,4] triazole |
| 37 | 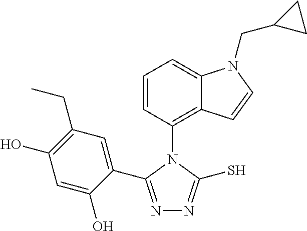 [SMILES: CCc1cc(-c2nnc(S)n2-c2cccc3c2ccn3CC2CC2)c(O)cc1O] | 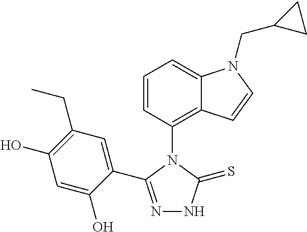 [SMILES: CCc1cc(-c2n[nH]c(=S)n2-c2cccc3c2ccn3CC2CC2)c(O)cc1O] | 3-(2,4-dihydroxy-5- cyclopropyl-phenyl)- 4-(1-(1- methylcyclopropyl)- indol-4-yl)-5- mercapto-[1,2,4] triazole |
| 38 | 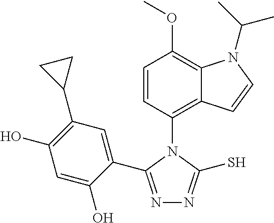 [SMILES: COc1ccc(-n2c(S)nnc2-c2cc(C3CC3)c(O)cc2O)c2ccn(C(C)C)c12] | 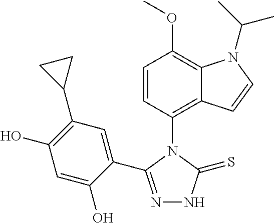 [SMILES: COc1ccc(-n2c(-c3cc(C4CC4)c(O)cc3O)n[nH]c2=S)c2ccn(C(C)C)c12] | 3-(2,4-dihydroxy-5- cyclopropyl-phenyl)- 4-(1-isopropyl-7- methoxy-indol-4-yl)- 5-mercapto-[1,2,4] triazole |
| 39 | 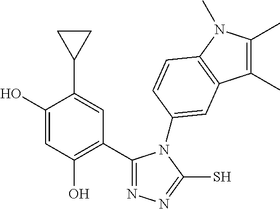 [SMILES: Cc1c(C)n(C)c2ccc(-n3c(S)nnc3-c3cc(C4CC4)c(O)cc3O)cc12] | 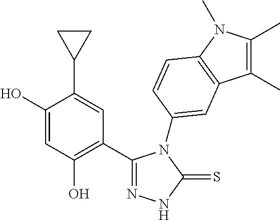 [SMILES: Cc1c(C)n(C)c2ccc(-n3c(-c4cc(C5CC5)c(O)cc4O)n[nH]c3=S)cc12] | 3-(2,4-dihydroxy-5- cyclopropyl-phenyl)- 4-(1,2,3-trimethyl- indol-5-yl)-5- mercapto-[1,2,4] triazole |
| 40 | 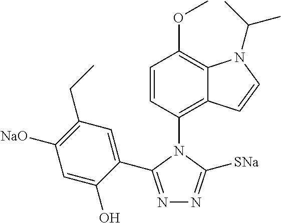 | | 3-(2,4-dihydroxy-5- ethyl-phenyl)-4-(1- isopropyl-7-methoxy- indol-4-yl)-5- mercapto-[1,2,4] triazole disodium salt |
| 41 | 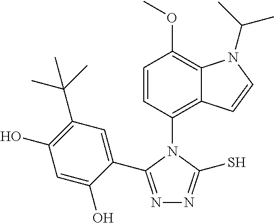 [SMILES: COc1ccc(-n2c(S)nnc2-c2cc(C(C)(C)C)c(O)cc2O)c2ccn(C(C)C)c12] | 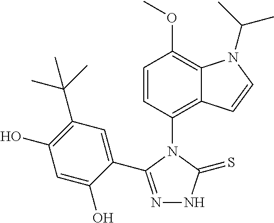 [SMILES: COc1ccc(-n2c(-c3cc(C(C)(C)C)c(O)cc3O)n[nH]c2=S)c2ccn(C(C)C)c12] | 3-(2,4-dihydroxy-5- tert-butyl-phenyl)-4- (1-isopropyl-7- methoxy-indol-4-yl)- 5-mercapto-[1,2,4] triazole |
| 42 | 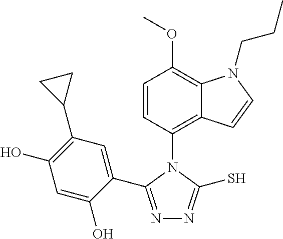 [SMILES: CCCn1ccc2c(-n3c(S)nnc3-c3cc(C4CC4)c(O)cc3O)ccc(OC)c21] | 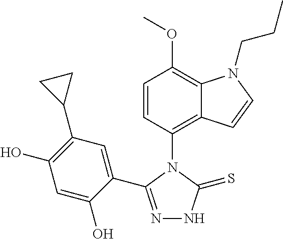 [SMILES: CCCn1ccc2c(-n3c(-c4cc(C5CC5)c(O)cc4O)n[nH]c3=S)ccc(OC)c21] | 3-(2,4-dihydroxy-5- cyclopropyl-phenyl)- 4-(1-propyl-7- methoxy-indol-4-yl)- 5-mercapto-[1,2,4] triazole |
| 43 | 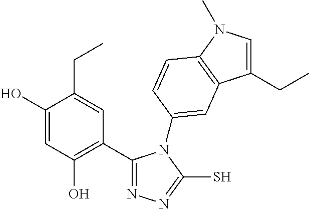 [SMILES: CCc1cc(-c2nnc(S)n2-c2ccc3c(c2)c(CC)cn3C)c(O)cc1O] | 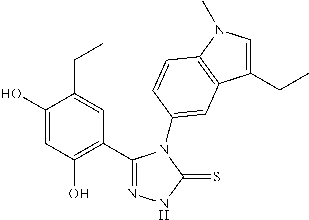 [SMILES: CCc1cc(-c2n[nH]c(=S)n2-c2ccc3c(c2)c(CC)cn3C)c(O)cc1O] | 3-(2,4-dihydroxy-5- ethyl-phenyl)-4-(1- methyl-3-ethyl-indol- 5-yl)-5-mercapto- [1,2,4]triazole |
| 44 | 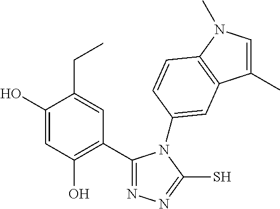 [SMILES: CCc1cc(-c2nnc(S)n2-c2ccc3c(c2)c(C)cn3C)c(O)cc1O] | 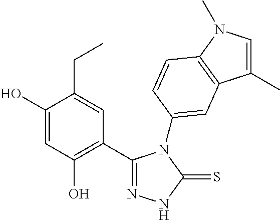 [SMILES: CCc1cc(-c2n[nH]c(=S)n2-c2ccc3c(c2)c(C)cn3C)c(O)cc1O] | 3-(2,4-dihydroxy-5- ethyl-phenyl)-4-(1,3- dimethyl-indol-5-yl)- 5-mercapto-[1,2,4] triazole |
| 45 | 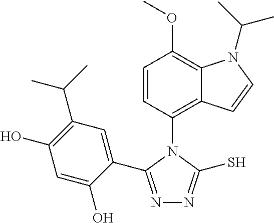 [SMILES: COc1ccc(-n2c(S)nnc2-c2cc(C(C)C)c(O)cc2O)c2ccn(C(C)C)c12] | 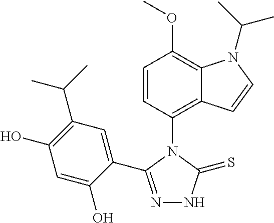 [SMILES: COc1ccc(-n2c(-c3cc(C(C)C)c(O)cc3O)n[nH]c2=S)c2ccn(C(C)C)c12] | 3-(2,4-dihydroxy-5- isopropyl-phenyl)-4- (1-isopropyl-7- methoxy-indol-4-yl)- 5-mercapto-[1,2,4] triazole |
| 46 | 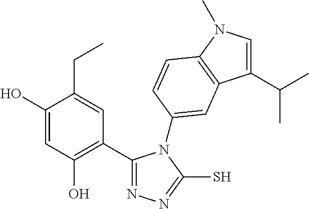 [SMILES: CCc1cc(-c2nnc(S)n2-c2ccc3c(c2)c(C(C)C)cn3C)c(O)cc1O] | 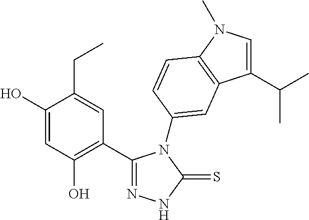 [SMILES: CCc1cc(-c2n[nH]c(=S)n2-c2ccc3c(c2)c(C(C)C)cn3C)c(O)cc1O] | 3-(2,4-dihydroxy-5- ethyl-phenyl)-4-(1- methyl-3-isopropyl- indol-5-yl)-5- mercapto-[1,2,4] triazole |
| 48 | 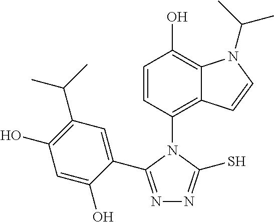 [SMILES: CC(C)c1cc(-c2nnc(S)n2-c2ccc(O)c3c2ccn3C(C)C)c(O)cc1O] | 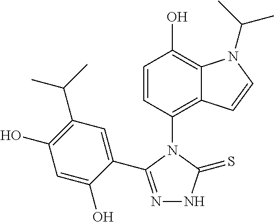 [SMILES: CC(C)c1cc(-c2n[nH]c(=S)n2-c2ccc(O)c3c2ccn3C(C)C)c(O)cc1O] | 3-(2,4-dihydroxy-5- ethyl-phenyl)-4-(1- isopropyl-7-hydroxy- indol-4-yl)-5- mercapto-[1,2,4] triazole |
| 49 | 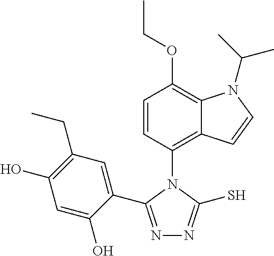 [SMILES: CCOc1ccc(-n2c(S)nnc2-c2cc(CC)c(O)cc2O)c2ccn(C(C)C)c12] | 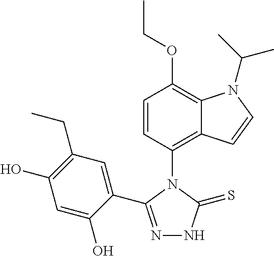 [SMILES: CCOc1ccc(-n2c(-c3cc(CC)c(O)cc3O)n[nH]c2=S)c2ccn(C(C)C)c12] | 3-(2,4-dihydroxy-5- ethyl-phenyl)-4-(1- isopropyl-7-ethoxy- indol-4-yl)-5- mercapto-[1,2,4] triazole |
| 50 | 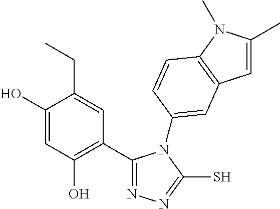 [SMILES: CCc1cc(-c2nnc(S)n2-c2ccc3c(c2)cc(C)n3C)c(O)cc1O] | 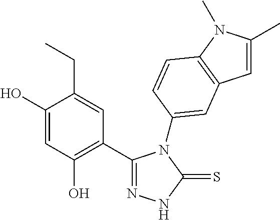 [SMILES: CCc1cc(-c2n[nH]c(=S)n2-c2ccc3c(c2)cc(C)n3C)c(O)cc1O] | 3-(2,4-dihydroxy-5- ethyl-phenyl)-4-(1,2- dimethyl-indol-5-yl)- 5-mercapto-[1,2,4] triazole |
| 51 | 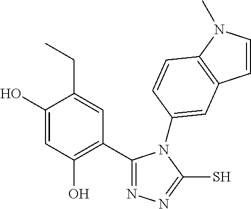 [SMILES: CCc1cc(-c2nnc(S)n2-c2ccc3c(ccn3C)c2)c(O)cc1O] | 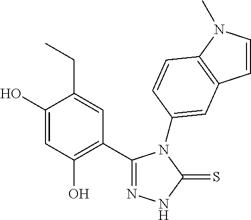 [SMILES: CCc1cc(-c2n[nH]c(=S)n2-c2ccc3c(ccn3C)c2)c(O)cc1O] | 3-(2,4-dihydroxy-5- ethyl-phenyl)-4-(N- methyl-indol-5-yl)-5- mercapto-[1,2,4] triazole |
| 55 | 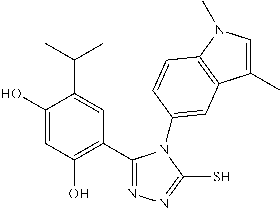 [SMILES: Cc1cn(C)c2ccc(-n3c(S)nnc3-c3cc(C(C)C)c(O)cc3O)cc12] | 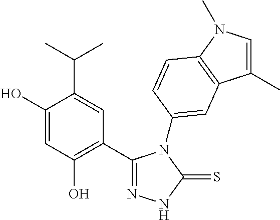 [SMILES: Cc1cn(C)c2ccc(-n3c(-c4cc(C(C)C)c(O)cc4O)n[nH]c3=S)cc12] | 3-(2,4-dihydroxy-5- isopropyl-phenyl)-4- (1,3-dimethyl-indol-5- yl)-5-mercapto-[1,2,4] triazole |
| 56 | 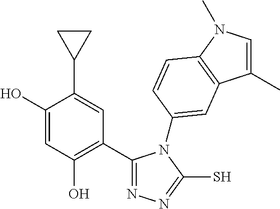 [SMILES: Cc1cn(C)c2ccc(-n3c(S)nnc3-c3cc(C4CC4)c(O)cc3O)cc12] | 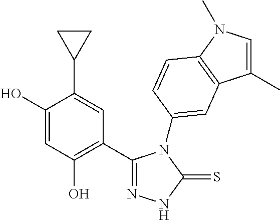 [SMILES: Cc1cn(C)c2ccc(-n3c(-c4cc(C5CC5)c(O)cc4O)n[nH]c3=S)cc12] | 3-(2,4-dihydroxy-5- cyclopropyl-phenyl)- 4-(1,3-dimethyl-indol- 5-yl)-5-mercapto- [1,2,4]triazole |
| 57 | 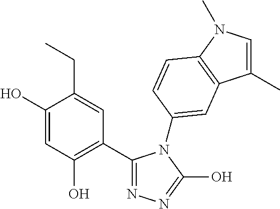 [SMILES: CCc1cc(-c2nnc(O)n2-c2ccc3c(c2)c(C)cn3C)c(O)cc1O] | 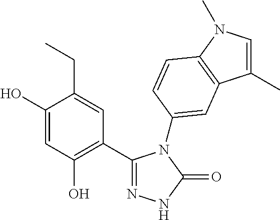 [SMILES: CCc1cc(-c2n[nH]c(=O)n2-c2ccc3c(c2)c(C)cn3C)c(O)cc1O] | 3-(2,4-dihydroxy-5- ethyl-phenyl)-4-(1,3- dimethyl-indol-5-yl)- 5-hydroxy-[1,2,4] triazole |
| 58 | 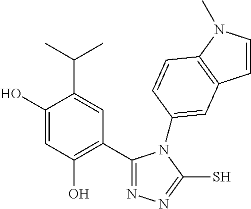 [SMILES: CC(C)c1cc(-c2nnc(S)n2-c2ccc3c(ccn3C)c2)c(O)cc1O] | 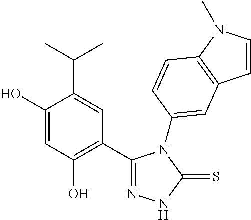 [SMILES: CC(C)c1cc(-c2n[nH]c(=S)n2-c2ccc3c(ccn3C)c2)c(O)cc1O] | 3-(2,4-dihydroxy-5- isopropyl-phenyl)-4- (N-methyl-indol-5- yl)-5-mercapto-[1,2,4] triazole |
| 59 | 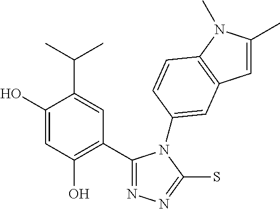 [SMILES: Cc1cc2cc(-n3c(S)nnc3-c3cc(C(C)C)c(O)cc3O)ccc2n1C] | 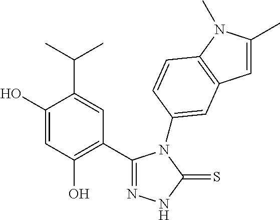 [SMILES: Cc1cc2cc(-n3c(-c4cc(C(C)C)c(O)cc4O)n[nH]c3=S)ccc2n1C] | 3-(2,4-dihydroxy-5- isopropyl-phenyl)-4- (1,2-dimethyl-indol-5- yl)-5-mercapto-[1,2,4] triazole |
| 60 | 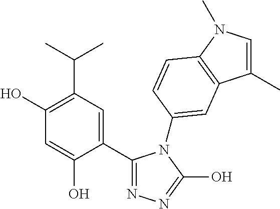 [SMILES: Cc1cn(C)c2ccc(-n3c(O)nnc3-c3cc(C(C)C)c(O)cc3O)cc12] | 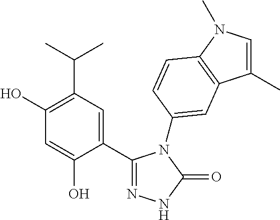 [SMILES: Cc1cn(C)c2ccc(-n3c(-c4cc(C(C)C)c(O)cc4O)n[nH]c3=O)cc12] | 3-(2,4-dihydroxy-5- isopropyl-phenyl)-4- (1,3-dimethyl-indol-5- yl)-5-hydroxy-[1,2,4] triazole |
| 62 | 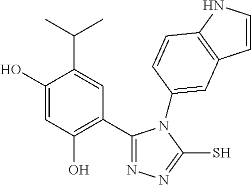 [SMILES: CC(C)c1cc(-c2nnc(S)n2-c2ccc3[nH]ccc3c2)c(O)cc1O] | 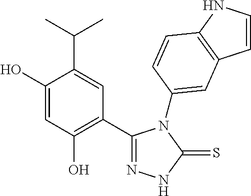 [SMILES: CC(C)c1cc(-c2n[nH]c(=S)n2-c2ccc3[nH]ccc3c2)c(O)cc1O] | 3-(2,4-dihydroxy-5- isopropyl-phenyl)-4- (1H-indol-5-yl)-5- mercapto-[1,2,4] triazole |
| 63 | 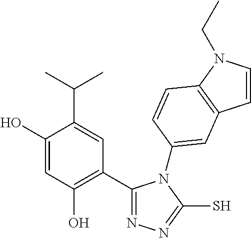 [SMILES: CCn1ccc2cc(-n3c(S)nnc3-c3cc(C(C)C)c(O)cc3O)ccc21] | 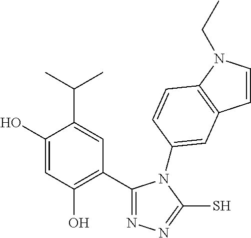 [SMILES: CCn1ccc2cc(-n3c(S)nnc3-c3cc(C(C)C)c(O)cc3O)ccc21] | 3-(2,4-dihydroxy-5- isopropyl-phenyl)-4- (1-ethyl-indol-5-yl)-5- mercapto-[1,2,4] triazole |
| 64 | 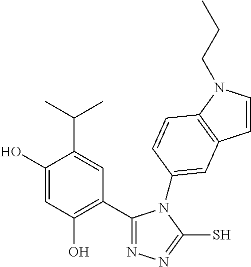 [SMILES: CCCn1ccc2cc(-n3c(S)nnc3-c3cc(C(C)C)c(O)cc3O)ccc21] | 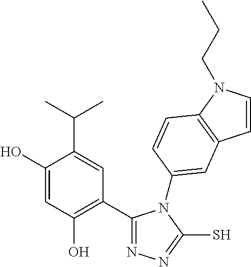 [SMILES: CCCn1ccc2cc(-n3c(S)nnc3-c3cc(C(C)C)c(O)cc3O)ccc21] | 3-(2,4-dihydroxy-5- isopropyl-phenyl)-4- (1-propyl-indol-5-yl)- 5-mercapto-[1,2,4] triazole |
| 65 | 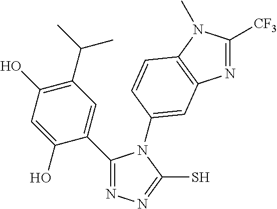 [SMILES: CC(C)c1cc(-c2nnc(S)n2-c2ccc3c(c2)nc(C(F)(F)F)n3C)c(O)cc1O] | 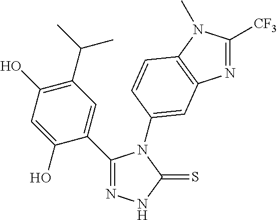 [SMILES: CC(C)c1cc(-c2n[nH]c(=S)n2-c2ccc3c(c2)nc(C(F)(F)F)n3C)c(O)cc1O] | 3-(2,4-dihydroxy-5- isopropyl-phenyl)-4- (1-methyl-2- trifluoromethyl- benzimidazol-5-yl)-5- mercapto-[1,2,4] triazole |
| 66 | 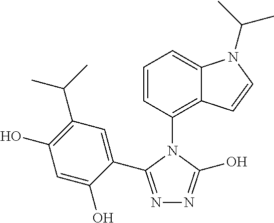 [SMILES: CC(C)c1cc(-c2nnc(O)n2-c2cccc3c2ccn3C(C)C)c(O)cc1O] | 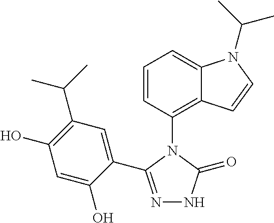 [SMILES: CC(C)c1cc(-c2n[nH]c(=O)n2-c2cccc3c2ccn3C(C)C)c(O)cc1O] | 3-(2,4-dihydroxy-5- isopropyl-phenyl)-4- (1-isopropyl-indol-4- yl)-5-hydroxy-[1,2,4] triazole |

TABLE 2
Compounds according to Formula (Ia)

| No. | Structure | Tautomeric structure | Name |
|---|---|---|---|
| 1a | 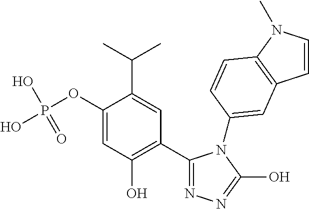 [SMILES: CC(C)c1cc(-c2nnc(O)n2-c2ccc3c(ccn3C)c2)c(O)cc1OP(=O)(O)O] | 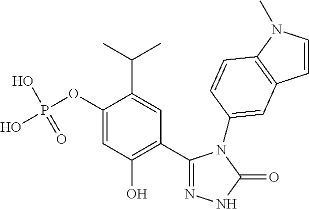 [SMILES: CC(C)c1cc(-c2n[nH]c(=O)n2-c2ccc3c(ccn3C)c2)c(O)cc1OP(=O)(O)O] | 5-hydroxy-4-(5- hydroxy-4-(1- methyl-1H- indol-5-yl)-4H- 1,2,4-triazol-3- yl)-2- isopropylphenyl dihydrogen phosphate |
| 2a | 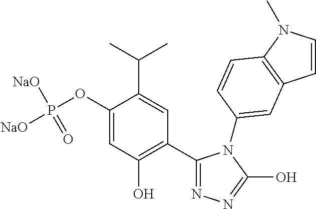 | 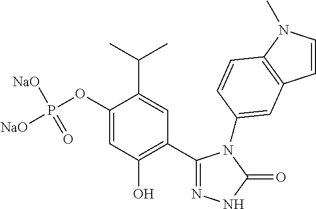 | sodium 5- hydroxy-4-(5- hydroxy-4-(1- methyl-1H- indol-5-yl)-4H- 1,2,4-triazol-3- yl)-2- isopropylphenyl phosphate |
| 3a | 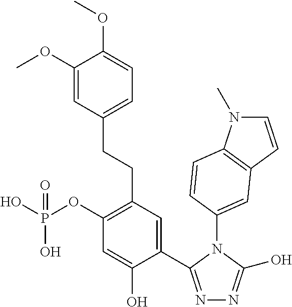 [SMILES: COc1ccc(CCc2cc(-c3nnc(O)n3-c3ccc4c(ccn4C)c3)c(O)cc2OP(=O)(O)O)cc1OC] | 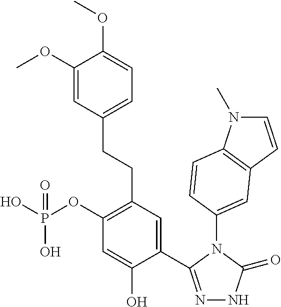 [SMILES: COc1ccc(CCc2cc(-c3n[nH]c(=O)n3-c3ccc4c(ccn4C)c3)c(O)cc2OP(=O)(O)O)cc1OC] | 2-(3,4- dimethoxy- phenethyl)- 5-hydroxy- 4-(5-hydroxy-4- (1-methyl-1H- indol-5-yl)-4H- 1,2,4-triazol-3- yl)phenyl dihydrogen phosphate |
| 4a | 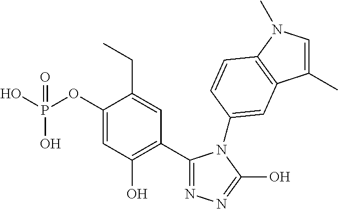 [SMILES: CCc1cc(-c2nnc(O)n2-c2ccc3c(c2)c(C)cn3C)c(O)cc1OP(=O)(O)O] | 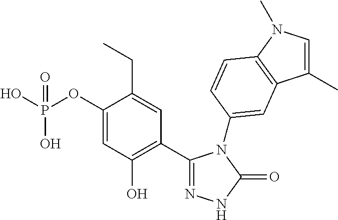 [SMILES: CCc1cc(-c2n[nH]c(=O)n2-c2ccc3c(c2)c(C)cn3C)c(O)cc1OP(=O)(O)O] | 4-(4-(1,3- dimethyl-1H- indol-5-yl)-5- hydroxy-4H- 1,2,4-triazol-3- yl)-2-ethyl-5- hydroxyphenyl dihydrogen phosphate |

Compounds used in the disclosed methods can be prepared according to methods disclosed in U.S. Application No. 2006-0167070 and WO2009/023211, the entire teachings of which are incorporated herein by reference.

Compounds of the invention typically can form a tautomeric structure as shown below and as exemplified by the tautomeric structures shown in Tables 1 and 2:

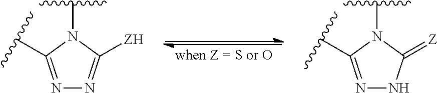

The invention also provides methods of treating, managing, or ameliorating breast cancer, particularly metastatic or hormonal therapy resistant breast cancer, or one or more symptoms thereof, said methods comprising administering to a subject in need thereof one or more compounds of the invention and one or more other therapies (e.g., one or more therapeutic agents that are currently being used, have been used, are known to be useful or in development for use in the treatment or amelioration of breast cancer, particularly metastatic or hormonal resistant breast cancer.

In one embodiment, the invention provides a method of treating a subject with breast cancer, wherein the subject is being or has been treated with a selective estrogen receptor modulator, comprising administering to the subject an effective amount of a selective estrogen receptor modulator and an effective amount of a triazolone compound represented by the structural formulae (I) or (Ia) or a compound in Table 1 or Table 2. In another embodiment, the invention also provides a method of treating a subject with metastatic breast cancer or hormonal therapy-resistant breast cancer, comprising administering to the subject an effective amount of a selective estrogen receptor modulator and an effective amount of a triazolone compound represented by the structural formulae (I) or (Ia) or a compound in Table 1 or Table 2. In yet another embodiment, the invention provides a method of modulating the response of hormonal therapy in a subject with breast cancer, comprising administering to the subject an effective amount of a selective estrogen receptor modulator and an effective amount of a triazolone compound represented by the structural formulae (I) or (Ia) or a compound in Table 1 or Table 2.

In another embodiment, the invention provides a method for treating a subject with breast cancer, comprising administering to the subject an effective amount of a selective estrogen receptor modulator and an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof.

In another embodiment, the invention provides a method for treating a subject with breast cancer, comprising administering to the subject an effective amount of tamoxifen and an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof.

In another embodiment, the invention provides a method for treating a subject with breast cancer, comprising administering to the subject an effective amount of a selective estrogen receptor modulator agent and an effective amount of 5-hydroxy-4-(5-hydroxy-4-(1-methyl-1H-indol-5-yl)-4H-1,2,4-triazol-3-yl)-2-isopropylphenyl dihydrogen phosphate dihydrogen phosphate, or a tautomer, or a pharmaceutically acceptable salt thereof.

In another embodiment, the invention provides a method for treating a subject with breast cancer, comprising administering to the subject an effective amount of tamoxifen and an effective amount of 5-hydroxy-4-(5-hydroxy-4-(1-methyl-1H-indol-5-yl)-4H-1,2,4-triazol-3-yl)-2-isopropylphenyl dihydrogen phosphate dihydrogen phosphate, or a tautomer, or a pharmaceutically acceptable salt thereof.

The therapeutic agents of the combination therapies of the invention can be administered sequentially or concurrently. In one embodiment, the administration of the HSP90 inhibitor and the selective estrogen receptor modulator are done concurrently. In another embodiment, the administration of the HSP90 inhibitor and the selective estrogen receptor modulator are done separately. In another embodiment, the administration of the HSP90 inhibitor and the selective estrogen receptor modulator are done sequentially. In another embodiment, the HSP90 inhibitor and the selective estrogen receptor modulator are administered in a single formulation. In another embodiment, the HSP90 inhibitor and the selective estrogen receptor modulator are administered intravenously, orally, transdermally, subcutaneously, mucosally, intramuscularly, intranasally, intrapulmonarily, parenterally, intrarectally or topically.

The therapeutic agents of the combination therapies can be administered to a subject, preferably a human subject, in the same pharmaceutical composition. In alternative embodiments, the therapeutic agents of the combination therapies can be administered concurrently to a subject in separate pharmaceutical compositions. The therapeutic agents may be administered to a subject by the same or different routes of administration.

In a specific embodiment, the combination therapies of the invention comprise one or more compounds and at least one other therapy which has the same mechanism of action as said compounds. In another specific embodiment, the combination therapies of the invention comprise one or more compounds of the invention and at least one other therapy which has a different mechanism of action than said compounds. In certain embodiments, the combination therapies of the present invention improve the therapeutic effect of one or more compounds of the invention by functioning together with the compounds to have an additive or synergistic effect. In certain embodiments, the combination therapies of the present invention reduce the side effects associated with the therapies. In certain embodiments, the combination therapies of the present invention reduce the effective dosage of one or more of the therapies.

In a specific embodiment, a pharmaceutical composition comprising one or more compounds of the invention is administered to a subject, preferably a human, to prevent, treat, manage, or ameliorate a proliferative disorder, such as cancer, or one or more symptom thereof. In accordance with the invention, pharmaceutical compositions of the invention may also comprise one or more other agents being used, have been used, or are known to be useful in the treatment or amelioration of breast cancer, particularly metastatic or hormonal therapy resistant breast cancer or a symptom thereof.

The invention provides methods for managing, treating or ameliorating breast cancer, particularly metastatic or hormonal therapy resistant breast cancer, or one or more symptoms thereof in a subject refractory (either completely or partially) to existing agent therapies for breast cancer, said methods comprising administering to said subject a dose of an effective amount of one or more compounds of the invention and a dose of an effective amount of one or more therapies. The invention also provides methods for treating, managing, or ameliorating breast cancer, particularly metastatic or hormonal therapy resistant breast cancer, or a symptom thereof by administering one or more compounds of the invention in combination with any other therapy(ies) to patients who have proven refractory to other therapies but are no longer on these therapies.

The compounds of the invention and/or other therapies can be administered to a subject by any route known to one of skill in the art. Examples of routes of administration include, but are not limited to, parenteral, e.g., intravenous, intradermal, subcutaneous, oral (e.g., inhalation), intranasal, transdermal (topical), transmucosal, and rectal administration.

The present invention provides compositions for the treatment, and amelioration of breast cancer, particularly metastatic or hormonal resistant breast cancer. In a specific embodiment, a composition comprises one or more compounds of the invention, or a pharmaceutically acceptable salt, thereof. In another embodiment, a composition of the invention comprises one or more therapeutic agents other than a compound of the invention, or a pharmaceutically acceptable salt. In another embodiment, a composition of the invention comprises one or more compounds of the invention, or a pharmaceutically acceptable salt thereof, and one or more other therapeutic agents. In another embodiment, the composition comprises a compound of the invention, or a pharmaceutically acceptable salt, thereof, and a pharmaceutically acceptable carrier, diluent or excipient.

In a preferred embodiment, a composition of the invention is a pharmaceutical composition or a single unit dosage form. Pharmaceutical compositions and dosage forms of the invention comprise one or more active ingredients in relative amounts and formulated in such a way that a given pharmaceutical composition or dosage form can be used to treat breast cancer, particularly metastatic or hormonal therapy resistant breast cancer. Preferred pharmaceutical compositions and dosage forms comprise a compound of any one of formulae (I) or (Ia) or a compound in Table 1 or 2, or a pharmaceutically acceptable thereof, optionally in combination with one or more additional active agents.

A pharmaceutical composition of the invention is formulated to be compatible with its intended route of administration. Examples of routes of administration include, but are not limited to, parenteral, e.g., intravenous, intradermal, subcutaneous, oral (e.g., inhalation), intranasal, transdermal (topical), transmucosal, and rectal administration. In a specific embodiment, the composition is formulated in accordance with routine procedures as a pharmaceutical composition adapted for intravenous, subcutaneous, intramuscular, oral, intranasal or topical administration to human beings. In a preferred embodiment, a pharmaceutical composition is formulated in accordance with routine procedures for subcutaneous administration to human beings.

Single unit dosage forms of the invention are suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form suitable for mucosal administration may contain a smaller amount of active ingredient(s) than an oral dosage form used to treat the same indication. This aspect of the invention will be readily apparent to those skilled in the art. See, e.g., Remington's Pharmaceutical Sciences (1990) 18th ed., Mack Publishing, Easton Pa.

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms.

The suitability of a particular excipient may also depend on the specific active ingredients in the dosage form. For example, the decomposition of some active ingredients can be accelerated by some excipients such as lactose, or when exposed to water. Active ingredients that comprise primary or secondary amines (e.g., N-desmethylvenlafaxine and N,N-didesmethylvenlafaxine) are particularly susceptible to such accelerated decomposition. Consequently, this invention encompasses pharmaceutical compositions and dosage forms that contain little, if any, lactose. As used herein, the term “lactose-free” means that the amount of lactose present, if any, is insufficient to substantially increase the degradation rate of an active ingredient. Lactose-free compositions of the invention can comprise excipients that are well known in the art and are listed, for example, in the U.S. Pharmocopia (USP)SP(XXI)/NF (XVI). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen (1995) Drug Stability: Principles & Practice, 2d. Ed., Marcel Dekker, NY, N.Y., 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizer” include, but are not limited to, antioxidants such as ascorbic acid, pH buffers, or salt buffers.

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences (1990) 18th ed., Mack Publishing, Easton Pa.

Typical oral dosage forms of the invention are prepared by combining the active ingredient(s) in an admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. One specific binder is a mixture of microcrystalline cellulose and sodium carboxymethyl cellulose sold as AVICEL RC-581. Suitable anhydrous or low moisture excipients or additives include AVICEL-PH-103J and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

A particular extended release formulation of this invention comprises a therapeutically or prophylactically effective amount of a compound of any one of formulae (I) or (Ia) or a compound in Table 1 or 2, or a pharmaceutically acceptable salt, in spheroids which further comprise microcrystalline cellulose and, optionally, hydroxypropylmethyl-cellulose coated with a mixture of ethyl cellulose and hydroxypropylmethylcellulose. Such extended release formulations can be prepared according to U.S. Pat. No. 6,274,171, the entirely of which is incorporated herein by reference.

A specific controlled-release formulation of this invention comprises from about 6% to about 40% a compound of formulae (I) or (Ia) or a compound in Table for 2, or a pharmaceutically acceptable salt, solvate, hydrate, clathrate, or prodrug thereof, by weight, about 50% to about 94% microcrystalline cellulose, NF, by weight, and optionally from about 0.25% to about 1% by weight of hydroxypropyl-methylcellulose, USP, wherein the spheroids are coated with a film coating composition comprised of ethyl cellulose and hydroxypropylmethylcellulose.

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences (1980 & 1990) 16th and 18th eds., Mack Publishing, Easton Pa. and Introduction to Pharmaceutical Dosage Forms (1985) 4th ed., Lea & Febiger, Philadelphia. Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences (1980 & 1990) 16th and 18th eds., Mack Publishing, Easton Pa.

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

The amount of the compound or composition of the invention which will be effective in the prevention, treatment, management, or amelioration of a proliferative disorder, such as breast cancer, or one or more symptoms thereof, will vary with the nature and severity of the disease or condition, and the route by which the active ingredient is administered. The frequency and dosage will also vary according to factors specific for each patient depending on the specific therapy (e.g., therapeutic or prophylactic agents) administered, the severity of the disorder, disease, or condition, the route of administration, as well as age, body, weight, response, and the past medical history of the patient. Effective doses may be extrapolated from dose-response curves derived from in vitro or animal model test systems. Suitable regiments can be selected by one skilled in the art by considering such factors and by following, for example, dosages reported in the literature and recommended in the Physician's Desk Reference (57th ed., 2003).

Exemplary doses of a small molecule include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

In general, the recommended daily dose range of a compound of the invention for the conditions described herein lie within the range of from about 0.01 mg to about 1000 mg per day, given as a single once-a-day dose preferably as divided doses throughout a day. In one embodiment, the daily dose is administered twice daily in equally divided doses. Specifically, a daily dose range should be from about 5 mg to about 500 mg per day, more specifically, between about 10 mg and about 200 mg per day. In managing the patient, the therapy should be initiated at a lower dose, perhaps about 1 mg to about 25 mg, and increased if necessary up to about 200 mg to about 1000 mg per day as either a single dose or divided doses, depending on the patient's global response. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with individual patient response.

Different therapeutically effective amounts may be applicable for different proliferative disorders, as will be readily known by those of ordinary skill in the art. Similarly, amounts sufficient to prevent, manage, treat or ameliorate such proliferative disorders, but insufficient to cause, or sufficient to reduce, adverse effects associated with the compounds of the invention are also encompassed by the above described dosage amounts and dose frequency schedules. Further, when a patient is administered multiple dosages of a compound of the invention, not all of the dosages need be the same. For example, the dosage administered to the patient may be increased to improve the prophylactic or therapeutic effect of the compound or it may be decreased to reduce one or more side effects that a particular patient is experiencing.

In a specific embodiment, the dosage of the composition of the invention or a compound of the invention administered to prevent, treat, manage, or ameliorate a proliferative disorders, such as cancer, or one or more symptoms thereof in a patient is 150 μg/kg, preferably 250 μg/kg, 500 μg/kg, 1 mg/kg, 5 mg/kg, 10 mg/kg, 25 mg/kg, 50 mg/kg, 75 mg/kg, 100 mg/kg, 125 mg/kg, 150 mg/kg, or 200 mg/kg or more of a patient's body weight. In another embodiment, the dosage of the composition of the invention or a compound of the invention administered to prevent, treat, manage, or ameliorate a proliferative disorders, such as cancer, or one or more symptoms thereof in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7m g, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg. The unit dose can be administered 1, 2, 3, 4 or more times daily, or once every 2, 3, 4, 5, 6 of 7 days, or once weekly, once every two weeks, once every three weeks or once monthly.

In general, the recommended dose range of a selective estrogen receptor modulator for the conditions described herein is its respective effective amount range as known in the art. The recommended dose range of a selective estrogen receptor modulator for the conditions described herein can be its respective maximum non-toxic amount dose. More particularly, the recommended daily dose range of a selective estrogen receptor modulator may be within the range of from about 0.01 mg to about 1000 mg per day, given as a single once-a-day dose preferably as divided doses throughout a day. Specifically, a daily dose range should be from about 5 mg to about 500 mg per day, more specifically, between about 10 mg and about 200 mg per day, even more specifically, between about 20 mg and about 100 mg. In managing the patient, the therapy should be initiated at a lower dose, perhaps about 1 mg to about 25 mg, and increased if necessary up to about 200 mg to about 1000 mg per day as either a single dose or divided doses, depending on the patient's global response. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with individual patient response.

In one embodiment, the selective estrogen receptor modulator (e.g., tamoxifen) is administered once daily schedule and the HSP inhibitor (e.g., Compound 1) is administered on week 1 and 2 (with a rest week after that) before beginning again. The amount of the HSP 90 inhibitor is adjusted according to tolerability and efficacy, as described above.

In one embodiment, the selective estrogen receptor modulator (e.g., tamoxifen) is administered once weekly schedule and the HSP inhibitor (e.g., Compound 1) is administered on week 1 and 2 (with a rest week after that) before beginning again. The amount of the HSP 90 inhibitor is adjusted according to tolerability and efficacy, as described above.

In another alternative, the selective estrogen receptor modulator is given daily (typical dose of 5 mg to 100 mg/day). Alternatively it is given once every two days. Doses range from 5 mg to 500 mg/day when given once two days. The dose of the HSP 90 inhibitor is commonly a full single agent dose (e.g., 200 mg/m2, or less, depending on tolerability, as described above.

Alternatively, the treatment cycle comprises weekly treatments for 2 weeks followed by a 1-week rest period. Treatment cycles will be repeated every 3 weeks. The HSP90 inhibitor is administered (150 mg/m² or 200 mg/m²) on Days 1 and 8 of each cycle and tamoxifen (20-100 mg/day) is administered daily, or every two days or every three days of each cycle. The treatment is repeated every three weeks.

The dosages of prophylactic or therapeutic agents other than compounds of the invention, which have been or are currently being used to prevent, treat, manage, or proliferative disorders, such as breast cancer, or one or more symptoms thereof can be used in the combination therapies of the invention. Preferably, dosages lower than those which have been or are currently being used to prevent, treat, manage, or ameliorate a proliferative disorder, or one or more symptoms thereof, are used in the combination therapies of the invention. The recommended dosages of agents currently used for the prevention, treatment, management, or amelioration of a proliferative disorders, such as breast cancer, or one or more symptoms thereof, can obtained from any reference in the art including, but not limited to, Hardman et al., eds., 1996, Goodman & Gilman's The Pharmacological Basis Of Basis Of Therapeutics 9^{th} Ed, Mc-Graw-Hill, New York; Physician's Desk Reference (PDR) 57^{th} Ed., 2003, Medical Economics Co., Inc., Montvale, N.J., which are incorporated herein by reference in its entirety.

In certain embodiments, when the compounds of the invention are administered in combination with another therapy, the therapies (e.g., prophylactic or therapeutic agents) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In one embodiment, two or more therapies (e.g., prophylactic or therapeutic agents) are administered within the same patent visit.

In certain embodiments, one or more compounds of the invention and one or more other the therapies (e.g., therapeutic agents) are cyclically administered. Cycling therapy involves the administration of a first therapy (e.g., a first prophylactic or therapeutic agents) for a period of time, followed by the administration of a second therapy (e.g., a second prophylactic or therapeutic agents) for a period of time, followed by the administration of a third therapy (e.g., a third prophylactic or therapeutic agents) for a period of time and so forth, and repeating this sequential administration, i.e., the cycle in order to reduce the development of resistance to one of the agents, to avoid or reduce the side effects of one of the agents, and/or to improve the efficacy of the treatment.

In certain embodiments, administration of the same compound of the invention may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

In a specific embodiment, the invention provides a method of preventing, treating, managing, or ameliorating a proliferative disorders, such as cancer, or one or more symptoms thereof, said methods comprising administering to a subject in need thereof a dose of at least 150 μg/kg, preferably at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds of the invention once every day, preferably, once every 2 days, once every 3 days, once every 4 days, once every 5 days, once every 6 days, once every 7 days, once every 8 days, once every 10 days, once every two weeks, once every three weeks, or once a month. Alternatively, the dose can be divided into portions (typically equal portions) administered two, three, four or more times a day.

In another embodiment, the invention also provides a method for identifying an agent that inhibits tamoxifen-resistant breast cancer cells, the method comprising measuring the level of cellular estrogen receptor of a breast cancer cell in a culture in the presence or absence of tamoxifen and in the presence or absence of the agent, because the level of cellular estrogen receptor of a breast cancer cell is proportional to the tamoxifen resistance potential of the cell, and a significant amount of inhibition of the level of cellular estrogen receptor by the agent should indicate that the agent is effective to inhibit the tamoxifen resistant breast cancer cell. Particularly, using MCF-7 breast cancer cells, disruption of estrogen receptor function by compound I was monitored in the presence and absence of tamoxifen, and estrogen receptor protein levels, localization, transcriptional activating activity and DNA binding were measured using standard techniques. The net effect of combining tamoxifen with compound 1 dramatically limited the emergence of tamoxifen resistant clones in cell culture.

The invention is illustrated by the following example which is not intended to be limiting in any way.

### Example 1

### Compound 1 Enhances the Anti-Tumor Activity of Tamoxifen Against Human Breast Tumor Cells in a Mouse Xenograft Model

The human breast cancer cell line, MCF-7, was obtained from the American Type Culture Collection (Manassas, Va., USA) and grown according to the ATCC guidelines. MCF-7 xenografts were established in twenty nude mice (Charles River Laboratories, Wilmington, Mass., USA) supplemented with slow release estrogen pellets as previously described, using 5×10⁶ cells (1), and randomized into treatment groups. Ten days after cells were inoculated half of the animals were supplemented with slow release tamoxifen pellets.

To formulate Compound 1 in 10/18 DRD, stock solutions of the test article were prepared by dissolving the appropriate amounts of the compound in dimethyl sulfoxide (DMSO) by sonication in an ultrasonic water bath. Stock solutions were prepared weekly, stored at −20° C. and diluted fresh each day for dosing. A solution of 20% Cremophor RH40 (polyoxyl 40 hydrogenated castor oil; BASF Corp., Aktiengesellschaft, Ludwigshafen, Germany) in 5% dextrose in water (D5W; Abbott Laboratories, North Chicago, Ill., USA) was also prepared by first heating 100% Cremophor RH40 at 50-60° C. until liquefied and clear, diluting 1:5 with 100% D5W, reheating again until clear and then mixing well. This solution was stored at room temperature for up to 3 months prior to use. To prepare 10/18 DRD formulations for daily dosing, DMSO stock solutions were diluted 1:10 with 20% Cremophor RH40. The final 10/18 DRD formulation for dosing contained 10% DMSO, 18% Cremophor RH40, 3.6% dextrose, 68.4% water and the appropriate amount of test article. Animals were intravenously (i.v.) injected with this formulation at 10 mL per kg body weight on one day each week for a total of 3 doses, four days after tamoxifen pellets were implanted.

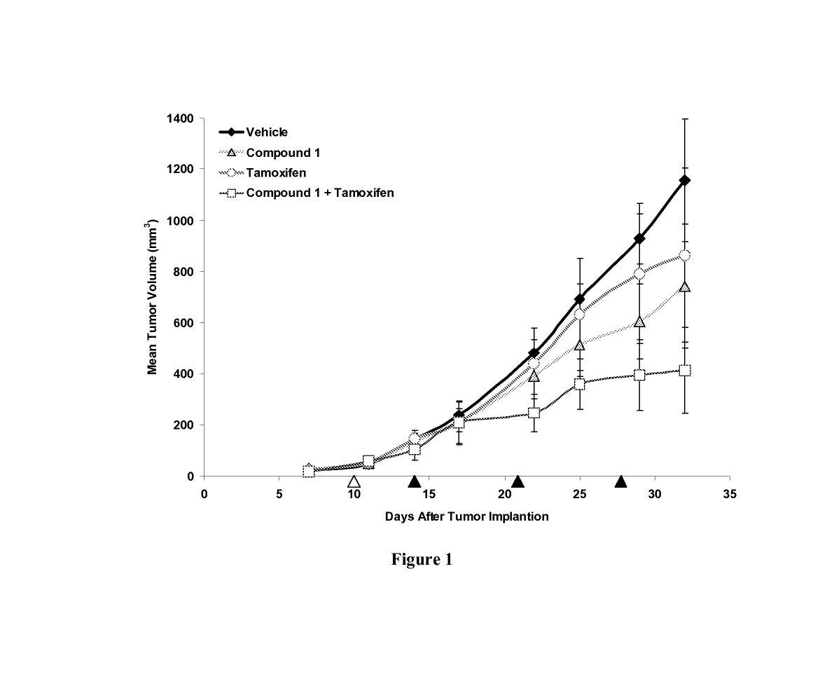

The ability of Compound 1 to enhance the in vivo anti-tumor activity of tamoxifen was investigated. As shown in FIG. 1, treatment with 180 mg/kg Compound 1 dosed 1 time per week, or slow release tamoxifen, resulted in moderate reductions in the growth rate of MCF-7 cells in nude mice. However, treatment with tamoxifen, combined with 180 mg/kg Compound 1 dosed 1 time per week, induced significant tumor regression. One of the animals in the Compound 1 alone cohort displayed signs of necrosis in the tail following the first dose and was removed from the study.

1-36. (canceled)

37. A method for treating a subject with breast cancer, metastatic breast cancer, or hormonal therapy-resistant breast cancer, comprising administering to the subject an effective amount of a selective estrogen receptor modulator and an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof.

38. The method of claim 37, wherein the selective estrogen receptor modulator is selected from the group consisting of tamoxifen, raloxifene, toremifene, fulvestrant, megestrol acetate, fluoxymesterone, and ethinyl estradiol.

39. The method of claim 38, wherein the estrogen receptor modulator treatment is tamoxifen or fulvestrant.

40. The method of claim 37, wherein the effective amount of the selective estrogen receptor modulator is within the range from about 0.01 mg/day to about 1000 mg/day.

41. The method of claim 37, wherein the effective amount of the triazolone compound is within the range from about 0.15 mg/kg to about 1000 mg/kg.

42. The method of claim 37, wherein the triazolone compound and the selective estrogen receptor modulator are administered simultaneously, or separately.

43. The method of claim 42, wherein the triazolone compound and the selective estrogen receptor modulator are administered at different times of day.

44. A method for treating a subject with breast cancer, metastatic breast cancer, or hormonal therapy-resistant breast cancer, comprising administering to the subject an effective amount of a selective estrogen receptor modulator agent and an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof.

45. The method of claim 44, wherein the selective estrogen receptor modulator is selected from the group consisting of tamoxifen, raloxifene, toremifene, fulvestrant, megestrol acetate, fluoxymesterone, and ethinyl estradiol.

46. The method of claim 45, wherein the estrogen receptor modulator treatment is tamoxifen or fulvestrant.

47. The method of claim 44, wherein the effective amount of the selective estrogen receptor modulator is within the range from about 0.01 mg/day to about 1000 mg/day.

48. The method of claim 44, wherein the effective amount of the triazolone compound is within the range from about 0.15 mg/kg to about 1000 mg/kg.

49. The method of claim 44, wherein the triazolone compound and the selective estrogen receptor modulator are administered simultaneously, or separately.

50. The method of claim 49, wherein the triazolone compound and the selective estrogen receptor modulator are administered at different times of day.

51. A pharmaceutical combination comprising a selective estrogen receptor modulator and 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, and a pharmaceutically acceptable carrier.

52. The pharmaceutical combination of claim 51, wherein the selective estrogen modulator is selected from the group consisting of tamoxifen, raloxifene, toremifene, fulvestrant, megestrol acetate, fluoxymesterone, and ethinyl estradiol.

53. The pharmaceutical combination of claim 52, wherein the selective estrogen modulator is tamoxifen, or fulvestrant.

54. A pharmaceutical combination comprising a selective estrogen receptor modulator and 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, and a pharmaceutically acceptable carrier.

55. The pharmaceutical combination of claim 53, wherein the selective estrogen modulator is selected from the group consisting of tamoxifen, raloxifene, toremifene, fulvestrant, megestrol acetate, fluoxymesterone, and ethinyl estradiol.

56. The pharmaceutical combination of claim 55, wherein the selective estrogen modulator is tamoxifen, or fulvestrant.
